SEQUENCE LISTING (22 sequences)

SEQ ID NO: 1 - protein, 10 aa, Artificial sequence
  Synthesized sequence
KGGPKKKRKV

SEQ ID NO: 2 - protein, 8 aa, Artificial sequence
  Synthesized sequence
RRRRRRRR

SEQ ID NO: 3 - protein, 5 aa, Artificial sequence
  Synthesized sequence
RRRRR

SEQ ID NO: 4 - protein, 11 aa, Artificial sequence
  Synthesized sequence
YGRKKRRQRRR

SEQ ID NO: 5 - protein, 19 aa, Artificial sequence
  Synthesized sequence
AAVALLPAVLLALLAPKKK

SEQ ID NO: 6 - protein, 10 aa, Artificial sequence
  Synthesized sequence
KFFKFFKFFK

SEQ ID NO: 7 - protein, 16 aa, Artificial sequence
  Synthesized sequence
RQIKIWFQNRRMKWKK

SEQ ID NO: 8 - protein, 30 aa, Artificial sequence
  Synthesized sequence
RRRQRRKKRGGDIMGEWGNEIFGAIAGFLG

SEQ ID NO: 9 - protein, 10 aa, Artificial sequence
  Synthesized sequence
VQRKRQKLMP

SEQ ID NO: 10 - protein, 8 aa, Artificial sequence
  Synthesized sequence
GRKRKKRT

SEQ ID NO: 11 - protein, 23 aa, Artificial sequence
  Synthesized sequence
GIGKFLHSAKKFGKAFVGEIMNS

SEQ ID NO: 12 - protein, 21 aa, Artificial sequence
  Synthesized sequence
TRSSRAGLQFPVGRVHRLLRK

SEQ ID NO: 13 - protein, 20 aa, Artificial sequence
  Synthesized sequence
VDKGSYLPRPTPPRPIYNRN

SEQ ID NO: 14 - protein, 5 aa, Artificial sequence
  SITE (4)..(4): Synthesized sequence; Xaa at position No. 4 is D-phenylalanine
RGDXK

SEQ ID NO: 15 - protein, 28 aa, Artificial sequence
  Synthesized sequence
RRRRRRRREDIIRNIARHLAQVGDSMDR

SEQ ID NO: 16 - protein, 29 aa, Artificial sequence
  Synthesized sequence
RKKRRQRRRSNRELVVDFLSYKLSQKGYS

SEQ ID NO: 17 - protein, 10 aa, Artificial sequence
  NON_TER (1)..(1): Synthesized sequence; Palmitic acid connected to arginine at position No. 1
RRERRDLFTE

SEQ ID NO: 18 - protein, 10 aa, Artificial sequence
  NON_TER (1)..(1): Synthesized sequence; Palmitic acid connected to lysine at position No. 1
KTRRKMDRYD

SEQ ID NO: 19 - protein, 10 aa, Artificial sequence
  NON_TER (1)..(1): Synthesized sequence; Palmitic acid connected to glycine at position No. 1
GFFKRNRPPL

SEQ ID NO: 20 - protein, 7 aa, Artificial sequence
  Synthesized sequence
KVGFFKR

SEQ ID NO: 21 - protein, 7 aa, Artificial
  Synthesized sequence
CKGDWPC

SEQ ID NO: 22 - protein, 10 aa, Artificial sequence
  Synthesized sequence
VQRKRQKLMP

(19) United States
(12) Patent Application Publication
(10) Pub. No.: US 2011/0033883 A1
(43) Pub. Date: Feb. 10, 2011

(54) IMAGING DYES AND USE THEREOF

(76) Inventors: Lynda Cosgrave, Dublin (IE); Tia E. Keyes, Dublin (IE); Robert J. Forster, Dublin (IE); Yann Pellegrin, Dublin (IE); Marc Devocelle, Dublin (IE); Niamh Moran, Dublin (IE); Dermot Kenny, Dublin (IE)

Correspondence Address: JACOBSON HOLMAN PLLC, 400 SEVENTH STREET N.W., SUITE 600, WASHINGTON, DC, 20004, US

(21) Appl. No.: 12/735,181

(22) PCT Filed: Dec. 19, 2008
(86) PCT No.: PCT/IE2008/000122

§ 371 Date: Jun. 18, 2010

Related U.S. Application Data
(60) Provisional application No. 61/006,105, filed on Dec. 19, 2007.

(51) Int. Cl. C12Q 1/02

(52) U.S. Cl. 435/29

(57) ABSTRACT

Use of a metal complex having the formula: [M(L¹)a(L²)b(L³)c]-Xd-Pepe wherein M is a metal selected from osmium, ruthenium, rhodium, rhenium or copper; L¹, L², L are bidentate or tridentate heterocyclic ligands containing O and/or N and may be the same or different; a, b, c are integers between 0 to 3 and may be the same or different and wherein the sum of a+b+c is 2 or 3; X is a functional group for directly or indirectly covalently binding to Pep wherein the functional group for directly covalently binding to Pep is selected from: amine, carboxylic acid, thiol or azide reactive functionalities; Pep is a peptide; d and e are integers between 1 and 3 and are the same and wherein the integers for d and e are equal to or less than the sum of a+b+c; and wherein there is optionally a linker between X and Pep for imaging of a cell or cell derived biological sample.

### INTRODUCTION

The invention relates to imaging dyes and use thereof. In particular the invention relates to dyes for use in resonance Raman imaging.

Imaging dyes for research and medical applications are known for example http://probes.invitrogen.com/. Typically imaging dyes have an optical signal such as fluorescence or luminescence that allows the dyes to be detected. Problems associated with the common commercial imaging dyes include short lived fluorescence/luminescence; poor environmental sensitivity, for example the imaging dye cannot detect changes in the environment such as oxygen levels, pH, water content and the like; and dyes are prone to photobleaching and may have poor photostability under detection conditions.

The known imaging dyes may have one or more of these problems associated with them. The problem of short lived fluorescence/luminescence and/or poor photostability is particularly problematic as dyes can suffer from background luminescence interference and the relatively short time course over which the dyes can be detected limits dynamic studies.

Quantum dots and nanoparticles are now being explored as imaging dyes (Ruan et al; Michalet et al; Morris et al; Wang et al, and Raymo et al) and have been shown to be more resistant to photobleaching than conventional imaging dyes and they can also provide very high intensity luminescence (European Patent number EP1441982). However, the cytotoxicity of quantum dots and nanoparticles has raised concerns over the use of such dyes for imaging live tissues and biological samples. In addition, their relatively large size means their ability to diffuse through cellular structures can be very poor.

There is a need for an imaging dye for medical, biomedical and research applications that is photostable, relatively non toxic and overcomes the problems associated with conventional imaging dyes.

### STATEMENTS OF INVENTION

According to the invention there is provided the use of a metal complex having the formula:

[M(L¹)ₐ(L²)_{b}(L³)_{c}]-X_{d}-Pepₑ
wherein:
M is a metal selected from osmium, ruthenium, rhodium, rhenium or copper;
L¹, L², L³ are bidentate or tridentate heterocyclic ligands containing O and/or N and may be the same or different;
a, b, c are integers between 0 to 3 and may be the same or different and wherein the sum of a+b+c is 2 or 3;
X is a functional group for directly or indirectly covalently binding to Pep wherein the functional group for directly covalently binding to Pep is selected from: amine, carboxylic acid, thiol or azide reactive functionalities;
Pep is a peptide;
d and e are integers between 1 and 3 and are the same and wherein the integers for d and e are equal to or less than the sum of a+b+c; and
wherein there is optionally a linker between X and Pep
for imaging of a cell or cell derived biological sample.

The cell or cell derived biological sample may be imaged using resonance Raman imaging and/or mapping. The cell or cell derived biological sample may be imaged using fluorescent imaging. The cell or cell derived biological sample may be imaged using resonance Raman imaging and/or mapping and fluorescent imaging. The fluorescent imaging may be fluorescent lifetime imaging.

One or more of L¹, L², L³ may be selected from the group comprising: 2,2-bipyridyl (bpy), 2,2-biquinoline (biq), 4,7-diphenyl-1,10-phenathroline (dpp), 2,3-bis(2-pyridyl)pyrazine (dppz) and 2-(4-carboxyphenyl)imidazo[4,5-f][1,10]phenanthroline (piCH₂).

The group to provide amine functionality may be selected from one or more of carboxylate, active ester, acid halide or isothiocyanate functionalities. The active ester may be a succinimidyl ester and/or a hydroxybenzotriazole ester. The group to provide carboxylic acid functionality may be selected from one or both of amine or isothiocyanate functionalities. The group to provide thiol functionality may be selected from one or more of iodoacetamide, maleimide, alkyl halide or isothiocyanate functionalities. The group to provide azide reactive functionality may be an alkyne functionality.

The peptide may comprise up to 50 amino acids in length, such as up to 30 amino acids in length, for example up to 20 amino acids in length or up to 10 amino acids in length. The peptide may comprise a transmembrane delivery sequence. The peptide may comprise any one of the amino acid sequences of SEQ ID No. 1 to SEQ ID No. 22.

There may be a linker between X and Pep. The linker may be an aliphatic compound. The linker may comprise an aliphatic compound having at least 2 carbon atoms. The linker may comprise an aliphatic compound having from 2 to 10 carbon atoms. The linker may be saturated. The linker may comprise a functional carboxyl group. The linker may be a straight chain molecule. The linker may be a hexyl linker. The linker may be a beta alanine.

The cell or cell derived biological sample may comprise live cells. The cell or cell derived biological sample may comprise a tissue sample.

The metal complex may have a Stokes shift of at least 50 nm such as a Stokes shift of at least 100 nm, for example a Stokes shift of at least 150 nm.

The metal complex may be luminescent. The metal complex may have an excitation wavelength between 380 nm to 1300 nm.

The metal complex may be used for imaging environmental parameters of a cell or cell derived biological sample. The environmental parameters may be selected from one or more of oxygen concentration, pH, and redox state. The oxygen concentration of a cell or cell derived biological sample may be imaged using fluorescence lifetime imaging. The pH of a cell or cell derived biological sample may be imaged using resonance Raman imaging and/or mapping. The redox state of a cell or cell derived biological sample may be imaged using fluorescence lifetime imaging and/or resonance Raman imaging and/or mapping.

One of the standard techniques in biological imaging is fluorescence microscopy which includes confocal fluorescence microscopy. Although these techniques are termed “fluorescence” the techniques can be used to image fluorescence and/or phosphorescence. The metal complexes described herein phosphoresce, therefore the general term luminescence has been used with respect to their optical properties and in this regard the term “fluorescence” imaging or microscopy can be understood to mean luminescence imaging or microscopy.

The invention also relates to the use of the following complexes and the complexes per se:

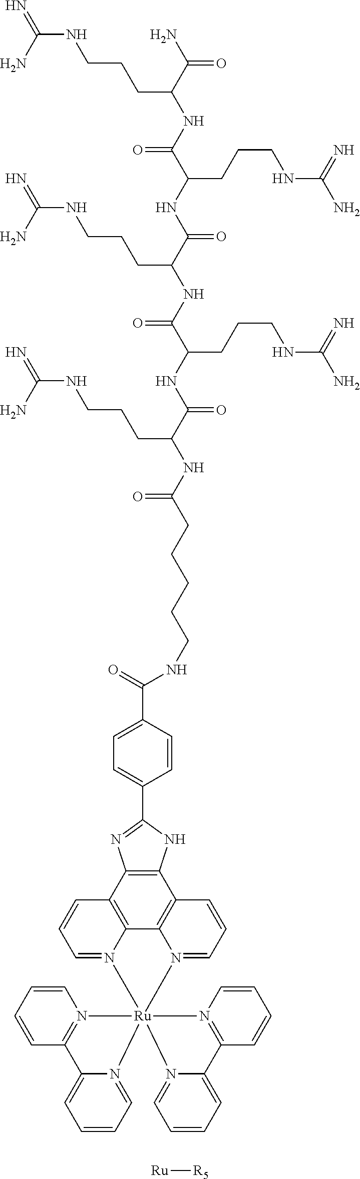

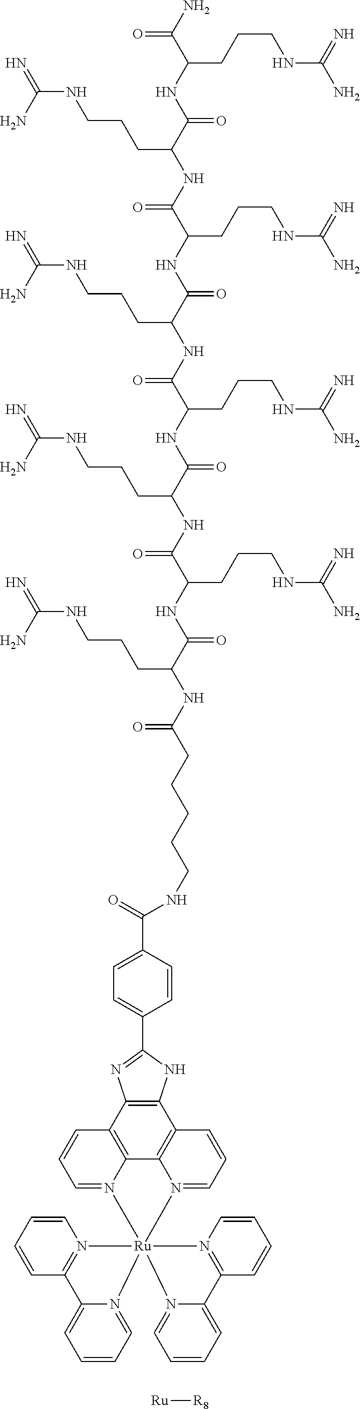

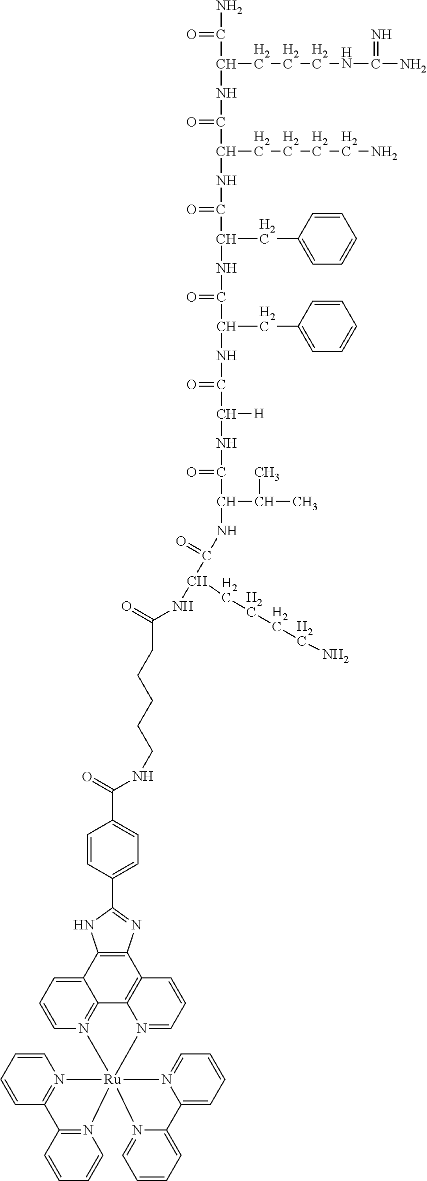

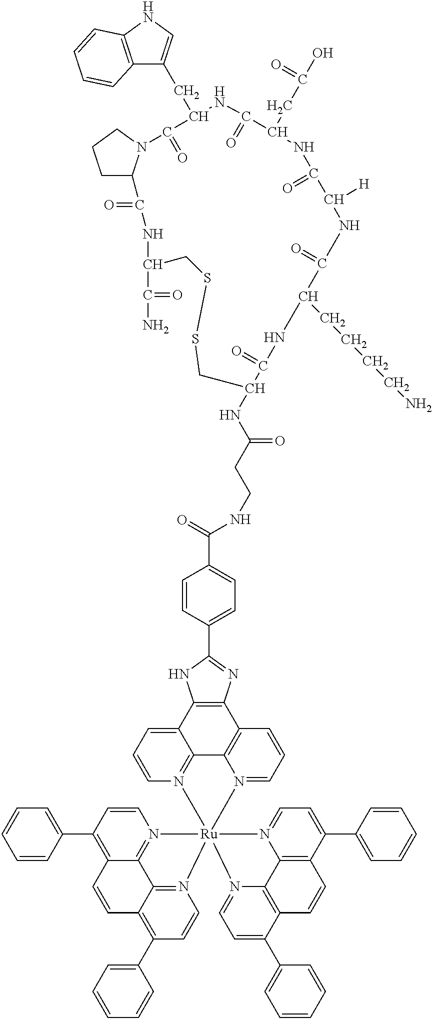

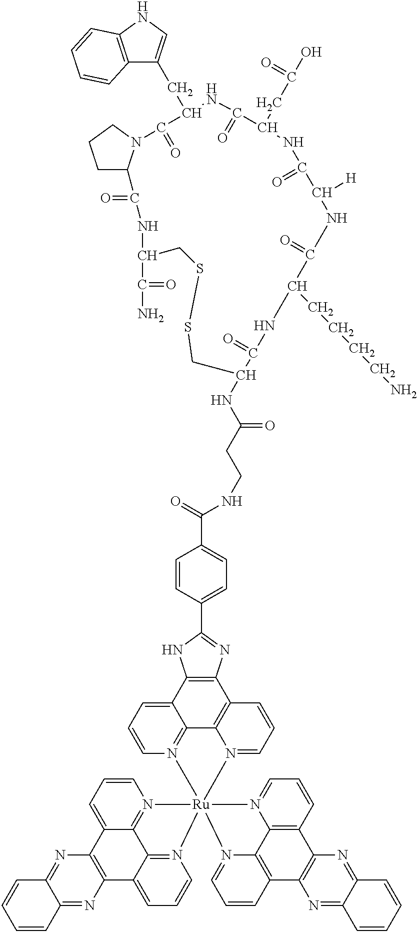

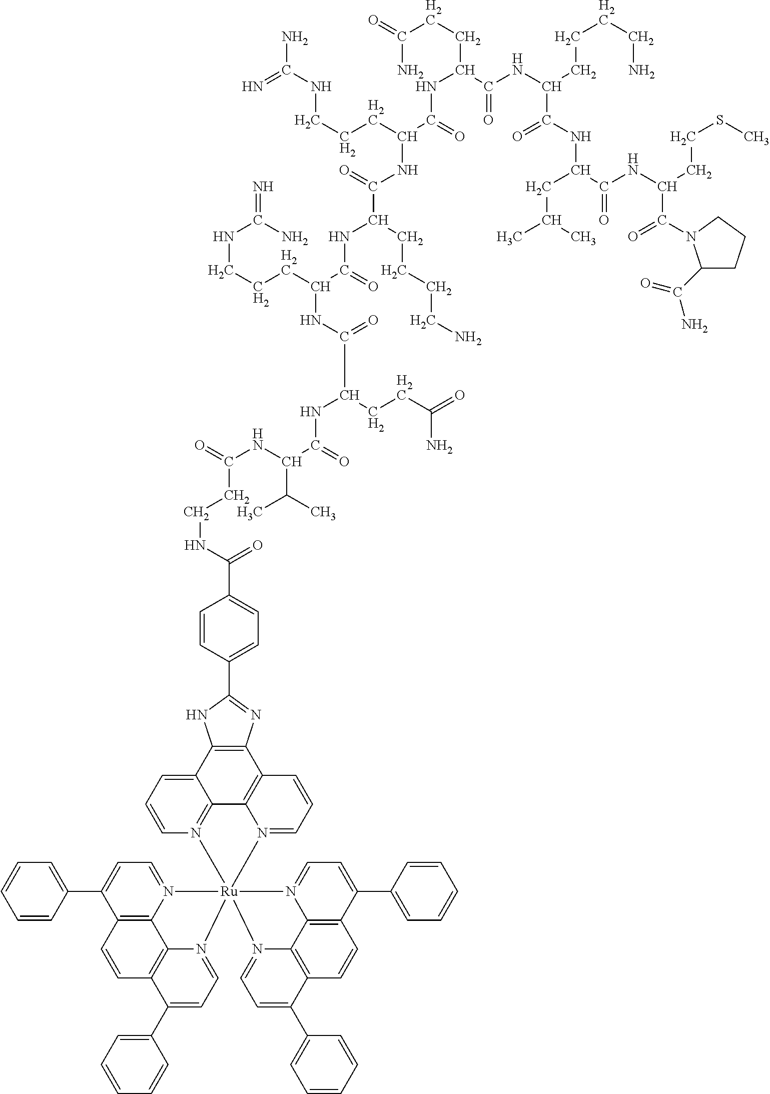

The invention further provides for a conjugate comprising:
a metal;
a ligand;
a linker; and
a peptide.

The conjugate may have the formula:

[M(L-L)ₐ(LX-pep)_{b}]
Wherein:
a and b are integers between 1 to 3, and may be the same or different
M=metal
L-L=bidentate, or tridentate heterocyclic ligand containing O or N or combinations thereof.

LX=bidentate or tridentate ligand of the type (L-L)-R—R¹—X where:
L-L is a bidentate, bi-heterocyclic ligand containing O or N or combinations thereof; this ligand may also contain surface active functionality.
R and R¹ are spacers; and
X is a functional group, which may be modified for protein.

Pep=peptide

The metal may form a luminescent complex. The metal may comprise a polypyridal unit. The metal may be selected from ruthenium, osmium, iridium, rhodium, rhenium or iron. The metal may have carboxy functionality.

L-L may be selected from the group comprising: 2,2-bipyridyl (bpy); 2,2-biquinoline (biq); 4,7-diphenyl-1,10-phenathroline (dpp); and 2,3-bis(2-pyridyl)pyrazine (dppz). The ligand and the linker may be the same entity.

The functional group of Lx may be capable of being covalently linked to an amino acid. The functional group may be a carboxyl or an amino or a thiol.

The linker may be an aliphatic compound such as an aliphatic compound with at least C₆. For example an aliphatic compound with C₆ to C₁₀. The linker may be saturated. The linker may comprise a pendant carboxyl group. The linker may be a straight chain molecule.

The peptide may comprise up to 20 amino acids. The peptide may comprise between 1 and 10 amino acids. The peptide may comprise naturally occurring amino acids. The peptide may comprise octa arginine (SEQ ID No 2). The peptide may comprise the sequence of SEQ ID No. 1.

The spacers of Lx may be aliphatic, alternatively, the spacers of Lx may be aromatic. The spacers of Lx may confer environmental sensitivity to the conjugate.

The properties of L-L may be modified to alter emission wavelength and/or sensitivity of the conjugate.

The conjugate may have an excitation wavelength between 380 nm to 1300 nm.

The conjugate may be transported across a cell membrane, for example the conjugate may be actively transported across a cell membrane. Alternatively, the conjugate may be passively transported across a cell membrane.

A conjugate may further comprise a targeting entity. For example the targeting entity may target the conjugate to an intracellular structure or organelle. Alternatively, the targeting entity may target the conjugate to an extracellular site.

The targeting entity and the peptide may be the same entity.

The invention further provides for a conjugate comprising the formula:

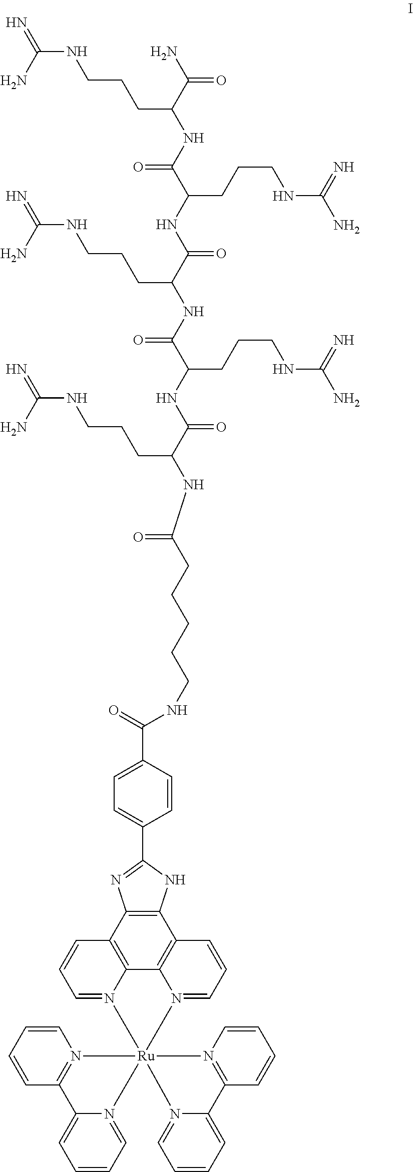

and a conjugate comprising the formula:

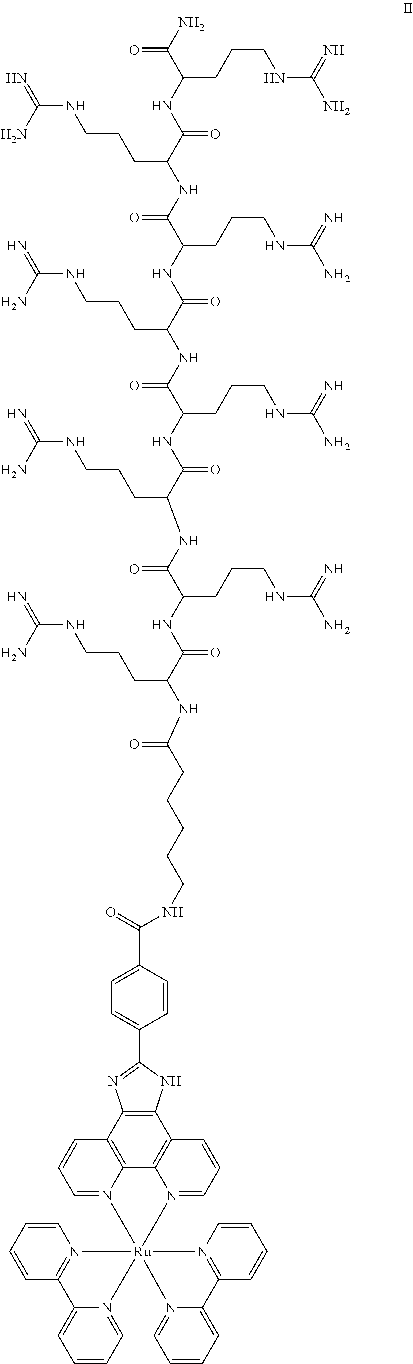

In a further aspect, the invention also provides for a dye comprising a conjugate as described herein.

In another aspect, the invention provides for a process of synthesising a conjugate as described herein comprising the steps of:
providing a peptide, or protein
linking a spacer to the peptide/protein;
providing a metal-ligand conjugate;
forming an inclusion complex of the metal ligand conjugate with a carrier, and
linking the inclusion complex thus formed to the spacer.

The spacer may be covalently linked to the peptide.

The inclusion complex may be covalently linked to the spacer.

The carrier may comprise a hydrophobic cavity. The carrier may be a carbohydrate, for example cyclodextrin.

The process may further comprise the step of synthesising the peptide. The peptide may further comprise at least one protective group. The peptide may contain an amine protection group, for example Fluorenyl-methoxy-carbonyl group. The process may further comprise the step of removing the amine protection group. The peptide may further comprise at least one side chain protection group. The side chain protection group may be pentamethyldihydrobenzofurane. The process may further comprise the step of removing the side chain protection group.

The peptide may be immobilised on a solid support. The peptide may be cleaved from the solid support when the dye conjugate has been formed. The peptide may be chemically cleaved. The process may further comprise the step of purifying the cleaved molecule.

The peptide may comprise up to 20 amino acids. For example, the peptide may comprise between 1 and 10 amino acids. The peptide may comprise naturally accruing amino acids. The peptide may comprise octa arginine (SEQ ID No 2). The peptide may comprise the sequence of SEQ ID No. 1.

The metal may be a luminophere, for example, the metal may be selected from: ruthenium, osmium, iridium, rhodium, rhenium or iron.

The ligand may be a bidentate, tridentate bi-heterocyclic ligand containing O or N donors. For example the ligand may be selected from the group comprising: 2,2-bipyridyl (bpy); 2,2-biquinoline (biq); 4,7-diphenyl-1,10-phenathroline (dpp); and 2,3-bis(2-pyridyl)pyrazine (dppz).

The spacer may be an aliphatic molecule. Alternatively, the spacer may be an aromatic molecule. The spacer may comprise a compound with C₆ to C₁₀. The spacer may be saturated. The spacer may comprise a pendant carboxyl group. The spacer may be a straight chain molecule. The spacer may be covalently linked to the peptide via a carboxy-amine interaction. The inclusion complex may be covalently linked to the spacer through an amide bond.

The conjugate may be a dye.

The invention further provides use of a conjugate as described herein for imaging biological samples. The imaging may be fluorescence based. The conjugate may be used for probing biological samples. The conjugate may be used for diagnosing a disease. The conjugate may be used for cellular Raman mapping or imaging.

The invention also provides a method of cellular mapping or imaging comprising the steps of:
introducing a conjugate as described herein into a sample containing cells;
exposing the sample to an excitation laser with a wavelength between 380 nm and 1300 nm; and
mapping or imaging the sample using Resonance Raman Spectroscopy.

The sample may contain chemically fixed cells, alternatively the sample may contain living cells.

Some of the advantages associated with the invention may include:
The dye conjugates described are long-lived and environmentally sensitive. The sensitivity of the dye conjugates can be tailored, e.g. to oxygen partial pressure, pH, water content etc., by altering the ligands and/or the metal ion of the dye conjugate.
Photobleaching: bleaching experiments, for example the data presented in FIG. 10, are a measure of the photostability of the dye conjugate under the types of continuous irradiation required for imaging. We have demonstrated that the dyes described herein are considerably less prone to photochemical bleach than common organic dyes. For example a ruthenium-polypyridyl complex covalently bound to an octa arginine peptide required approximately 20 minutes continuous irradiation for the dye to bleach to 50% of its initial intensity. This protein-dye conjugate significantly outperforms conventional organic dyes that bleach within 5 minutes under identical conditions. The dye conjugates described herein provide a much greater acquisition time which is a significant advantage to the microscopist/microbiologist.
The synthetic yields for coupling of the Rupic unit to amine functionalities are high. The dye conjugates are functionalised with a single, accessible group, allowing unequivocal reaction with nucleophilic functions on peptides or proteins. They do not contain any isomers or competing functional groups which can lower the synthetic yields of the labelling step and/or require protection (Fischer R, et al)
One or more protein/peptide component can be attached directly or indirectly (via a linker) to the metal-ligand complex so as to confer the ability to efficiently transfer passively across the cell membrane.
One or more protein/peptide component can be attached directly or indirectly (via a linker) to the metal-ligand complex so as to enable the protein-dye conjugate specifically target a receptor site within a protein, cell or tissue.
The dye conjugates of the invention are long-lived, intense, and environmentally sensitive, their environmental sensitivity can manifest as either a change in emission lifetime and/or intensity or a modification of the resonance Raman spectrum or both.
The dye conjugates, because of their optical properties, can be used as a single agent for resonance Raman and luminescence imaging.
The dye conjugates can be designed to passively and efficiently transfer across a cell membrane, and in some cases to selectively bind to specific receptors in proteins, cells and tissues. The dye conjugates are easily synthetically modified to change colour, lifetime, and environmental sensitivity and are highly resistant to photobleaching.

### BRIEF DESCRIPTION OF THE DRAWINGS

The invention will be more clearly understood from the following description of an embodiment thereof, given by way of example only, with reference to the accompanying drawings, in which:—

FIG. 1A is a schematic representation of a resin bead bearing amino functions;

FIG. 1B is a schematic representation of a resin bead bearing an amino group which is linked to a polypeptide. Cleavage of the amino linked peptide (amide terminated polypeptide) from the resin bead is effected by trifluoroacetic acid (TFA) (FIGS. 1A and 1B show schematically how the synthesis of peptide occurs using a resin bead synthesis procedure);

FIG. 1C is a schematic representation of a parent carboxylate functionalised complex [Ru(bpy)₂PicH₂]²⁺. The complex is amidated with an amino-group connected to an inert backbone and can be used to test the spectral properties of the complex. This carboxylate of the complex can be used for conjugation to a peptide or protein sequence;

FIG. 2 (A) is a graph showing the emission spectra of an aqueous solution of [Ru(bpy)₂PicH₂]²⁺ at pH 7.0 (solid line), Ru and [Ru(bpy)₂Pic]⁺ at pH 12.0 (dotted line). The inset is a graph showing the decay of the luminescence of a 10 μm of [Ru(bpy)₂PicH₂]²⁺ in degassed acetonitrile; (B) shows the absorbance spectrum of an aqueous solution of [Ru(bpy)₂PicH₂]²⁺ at pH 7.0 (solid line) and [Ru(bpy)₂Pic]⁺ at pH 12.0 (dotted line) with an insert showing the pH dependent changes to the UV spectrum monitored at 340 nm;

FIG. 3 shows cyclic voltammograms of [Ru(bpy)₂PicH₂]²⁺ at pH 7.0 (solid line) and [Ru(bpy)₂Pic]⁺ at pH 12.0 (dotted line) in 0.1M phosphate buffer. The working electrode is glassy carbon disk (2 mm diameter), the auxiliary electrode is platinum wire and the reference electrode is aqueous Ag/AgCl; The inset shows cyclic voltammograms of [Ru-LH]²⁺ in acetonitrile;

FIG. 4 is a resonance Raman Spectra of 1×10⁻³M [Ru(bpy)₂PicH₂](ClO₄)₂ in aqueous buffered solution as a function of pH at an excitation wavelength of 458.7 nm;

FIG. 5 is luminescence spectra as a function of pH for [Ru(bpy)₂PicH₂]²⁺ in phosphate buffered aqueous solution in the range pH 4 to 11 (A) and pH 4 to 0.5 (B). Inserts show the fit of the resulting data to obtain kᵢ;

FIG. 6 (A) shows the structure of [Ru(bpy)₂PicHR₈]ⁿ⁺ and (B) shows the pH dependent resonance Raman Spectroscopy of [Ru(bpy)₂PicHR₈]ⁿ⁺ in aqueous media at an excitation wavelength of 514 nm;

FIG. 7 (A) is a resonance Raman mapping image of a myeloma cell following exposure to [Ru(bpy)₂PicHR₈]ⁿ⁺. The map was generated using the vibrational mode 1480 cm⁻¹ and the background was taken at 1750 cm⁻¹ from the resonance Raman map of a Myeloma cell (B) is the white light image of the cell. (C), (D) and (E) are the resonance Raman spectra from [Ru(bpy)₂PicHR₈]ⁿ⁺ which has passively diffused through the cell at different sites around the cell. (E) shows the spectrum when the microscope is focussed on the background solution;

FIG. 8 is a graph showing electronic absorption and emission spectra for [Ru(bpy)₂(picHR₈)]²⁺ in buffered aqueous saline, pH 7, at an excitation wavelength of 450 nm;

FIG. 9 shows microscopy images of human blood platelets treated with the hybrid luminophore [Ru(bpy)₂PicHR₈]ⁿ⁺. (A) shows a phase contrast image, (B) shows a fluorescence confocal microscopy image and (C) is an overlay image of (A) and (B);

FIG. 10 is a graph showing a photobleaching experiment. The graphs plot the emission intensity vs time under continuous irradiation at 458 nm for [Ru(bpy)₂PicHR₈]ⁿ⁺ labelled human blood platelets;

FIG. 11 shows the effect of increasing water concentration on the luminescence intensity (A) and lifetime (B) of a 0.035 mM solution of [Ru(dppz)₂(PicH)]²⁺ in acetonitrile on addition of 50 μL (i.e. 2.79×10⁻³ moles) of deionised water. The use of this dye in resonance Raman imaging is shown. (C) shows the structure of [Ru(dppz)₂(PicH-R₈)]²⁺;

FIG. 12 is a resonance Raman mapping image of a myeloma cell following exposure to [Ru(dppz)₂(PicH-R₈)]²⁺. The map was generated using vibrational 1422 cm⁻¹ and the background around 1750 cm⁻¹ from the resonance Raman map of a Myeloma cell C and D the Resonance Raman spectra from [Ru(dppz)₂(PicH-R₈)]²⁺ which has passively diffused through the cell at the membrane and what is thought to be the nucleus of the cell. Differences in spectral features are due to different water content of each region;

FIG. 13 (A) is a UV/Vis absorption spectra of [Ru(bpy)₂(PicH-R₈)]²⁺ at different pHs. (B) is a graph showing the change of absorption at 350 nm (ππ* Pic) as a function of pH, the dots represents the pH titration data and the solid line represents the fit with the Henderson-Hasselbalch equation;

FIG. 14 (A) is a graph showing the dependence of fluorescence lifetime of [Ru(bpy)₂(picH-R₈)]²⁺ on pH in aerated phosphate buffered aqueous solution ★ and in deaerated phosphate buffered aqueous solution ; and (B) are Stern-Volmer plots of measuring the quenching of [Ru(bpy)₂(PicH-R₈)]²⁺ by oxygen in phosphate buffered aqueous solution at different pH;

FIG. 15 (A) is a resonance Raman spectra for [Ru(Pic)₃], [Ru(bpy)₂(PicH₂)]²⁺, and [Ru(bpy)₃] at 458 nm excitation, (B) is a resonance Raman spectra showing the pH dependence of emission lifetime of [Ru(bpy)₂(PicH₂)]²⁺;

FIG. 16 (A) are resonance Raman images (greyscale) obtained from resonance Raman mapping of myeloma cells (top) using the band at 1480 cm⁻¹, reflecting distribution of the dye throughout the cell (bottom) is a Raman intensity ratio map of pH sensitive and insensitive bands at 1622 and 1318 cm⁻¹ which reflects the distribution of regions of different pH around the cell. (B) (top) are graphs showing the ratio of peaks at different pH from the resonance Raman map fitted to a Henderson-Hasselbalch equation; and (bottom) are the regions of different pH extracted out of 16(A) bottom;

FIG. 17 (A) is a confocal luminescence intensity image of a myeloma cell incubated with [Ru(bpy)₂(picH-R₈)]²⁺ for 15 mins; (B) is a fluorescence lifetime image (FLIM) of the cell of (A); and (C) is a detailed FLIM image of the cell of (A), where regions of different lifetime are extracted;

FIG. 18 (A) are fluorescent images of a stained myeloma cell following 3 min and 6 min incubation with [Ru(bpy)₂(picH-R₈)]²⁺; (B) are images of a myeloma cell incubated with [Ru(bpy)₂(picH-R₈)]²⁺ for 20 mins and counter stained with DiOC which stains the mitochondria and cellular membranes; The left hand side of the first three panels show the DiOC fluorescence only and the right hand side show the luminescence from the [Ru(bpy)₂(picH-R₈)]²⁺ (DiOC is filtered out). The bottom left Fig. shows show the DiOC fluorescence only and the bottom right panel shows [Ru(bpy)₂(picH-R₈)]²⁺ and DiOC luminescence superimposed (C) are images of a myeloma cell incubated with [Ru(bpy)₂(picH-R₈)]²⁺ for 20 mins and counter stained Sytox green which only enters cells with a compromised cell membrane; The left shows the image in which emission from Sytox green is shown with [Ru(bpy)₂(picH-R₈)]²⁺ filtered out, no luminescence is observed confirming the cell is living and the left shows the [Ru(bpy)₂(picH-R₈)]²⁺ luminescence image with Sytox green filtered out;

FIG. 19 (A) is an emission spectra of [Ru(dppz)₂PicH₂]²⁺ (top), [Ru(dppz)₂(PICH-R₅)]²⁺ (middle), and [Ru(dppz)₂(PicH-R₈)]²⁺ (bottom) in 9:1 acetonitrile:DMSO (all solutions were absorbance matched); (B) is a UV/Vis absorbance spectra of [Ru(dppz)₂PICH₂]²⁺ (solid line), [Ru(dppz)₂(PicH-R₅)]²⁺ (dashed line), and [Ru(dppz)₂(PicH-R₈)]²⁺ (dotted line) in 9:1 acetonitrile:DMSO; and (C) is an emission lifetime spectra for [Ru(dppz)₂PicH₂]²⁺ in acetonitrile, 9:1 acetonitrile:DMSO and methanol;

FIG. 20 is an emission and lifetime (inset) spectra of [Ru(dppz)₂PICH₂]²⁺ in dry degassed acetonitrile after sequential addition of deionised water at room temperature. The most intense plot shows [Ru(dppz)₂PICH₂]²⁺ in dry degassed acetonitrile in the absence of water and subsequent spectra show the decreasing emission with 50 μL aliquot additions to the solution of the complex up to 1000 μL to 5 mL of solution. The spectra are adjusted for dilution;

FIG. 21 plots the effect of emission lifetime and intensity as a of [Ru(dppz)₂PICH₂]²⁺ in dry degassed acetonitrile as a function of water concentration (M);

FIG. 22 are emission spectra of [Ru(dppz)₂(PicH-R₈)]²⁺ (A); [Ru(dppz)₂(PicH-R₅)]²⁺ (B); and [Ru(dppz)₂(PicH₂)]²⁺ (C) over time (approximately 6 days) in the presence of DPPG liposomes (solid line); and absence of liposomes (just buffer at pH 7.4, dotted line). All solutions were absorbance matched and had the same phospholipids concentration. The inset of (C) shows the lifetime data for [Ru(dppz)₂(PicH-R₈)]²⁺ (circles); [Ru(dppz)₂(PicH-R₅)]²⁺ (diamonds); and [Ru(dppz)₂(PicH)]²⁺ (crosses) in DPPG liposomes;

FIG. 23 are resonance Raman intensity plots of a myeloma cell constructed from the intensity of the peak at 1593 cm⁻¹ in the spectrum of [Ru(dppz)₂(PICH-R₈)]²⁺ (bottom) and [Ru(dppz)₂(PICH₂)]²⁺ (top) after excitation at 488 nm. The greyscale bar indicates the relative resonance Raman signal intensity that decreases from top to bottom and the images on the right, indicating the different concentrations of dye in the cells. Images on the left are the white light images of the mapped cells;

FIG. 24 is a resonance Raman spectrum of [Ru(dppz)₂(PICH-R₈)]²⁺ (A) and [Ru(dppz)₂(PICH₂)]²⁺ (B) in a myeloma cell (taken from crosshairs on the cell map shown in FIG. 1023) (dotted line) and in pH 7.4 buffer (solid line);

FIG. 25 is a resonance Raman spectra of [Ru(dppz)₂(PicH-R₈)]²⁺ (solid line) and [Ru(dppz)₂(PicH)]²⁺ (dotted line) in pH 7.4 buffer;

FIG. 26 is a fluorescence lifetime image of (A) [Ru(dppz)₂(PicH₂)]²⁺ and (B) [Ru(dppz)₂(PicH-R₈)]²⁺ following 20 mins incubation, at 22° C., with myeloma cells in Tris/KCl buffer;

FIG. 27 is a normalised absorbance (left) and emission spectra (right) for [Cu(dop)₂]⁺ in ethanol, the insert shows the structure of dop;

FIG. 28 is a resonance Raman spectrum of [Cu(dop)₂]⁺ in KBr excited at 458 nm (absence of emission interference indicates that the Stokes shift is sufficient for coincident excitation for emission and Raman imaging);

FIG. 29 is a normalised absorbance and emission spectrum for [Ru(bpy)₂(biq)]⁺ in acetonitrile, where biq is 2,2-biquinoline;

FIG. 30 is a normalised absorbance and emission spectrum for [Os(bpy)₂(piCH₂)]²⁺ in water; and

FIG. 31 is an emission spectrum (left) and a confocal image at a excitation wavelength of 458 nm (right) of bovine aortic epithelial cells in the presence of [Ru(bpy)₂(pic-KVG)]ⁿ⁺ where KVG is KVGFFKR-NH₂ (SEQ ID No. 20).

### DETAILED DESCRIPTION

We describe dye-protein conjugates in which peptides/polypeptides/proteins (these terms are used interchangeably from hereon in) are covalently labelled with dyes based on transition metal complexes.

The protein-dye conjugate contains functionally distinct units, namely:

### Dye (Metal-Ligand Complex)

The dye may comprise a transition metal complex coordinated to tridentate or bidentate ligands to form a metal-ligand complex. The metal and/or ligands can be selected to tune the absorbance/emission spectra and/or environmental sensitivity of the dye. One or more of the ligands may contain a functional group through which a linker or a protein can be covalently bound to the metal-ligand complex. In the case where a peptide/protein is directly bound (not via a linker) to a metal-ligand complex the ligand may have an amine group, carboxylic acid group or thiol reactive functionality such as, carboxylate, amino, iodoacetamide, maleimide, active ester such as succinimidyl ester or hydroxybenzotriazole ester, an alkyl halide, an acid halide, isothiocyanates, azide or alkyne functionality that can be used to directly covalently bind a peptide/protein to the ligand. In the case where a protein is indirectly bound to a metal-ligand complex via a linker, the linker may have one or more of the functionalities listed above for the ligand.

The metal may be a transition metal which commonly forms luminescent complexes which exhibit large Stokes shifts, such as osmium, ruthenium, rhodium, rhenium or copper.

The ligand may be one or more of a bidentate or tridentate heterocyclic ligand containing O and/or N donors. For example, the ligand may be selected from one or more of 2,2-bipyridyl (bpy), 2,2-biquinoline (biq), 4,7-diphenyl-1,10-phenathroline (dpp), 2,3-bis(2-pyridyl)pyrazine (dppz) and 2-(4-carboxyphenyl)imidazo[4,5-f][1,10] phenanthroline (piCH₂).

The metal-ligand complexes of the invention have one or more of the following properties:
The electronically excited state may lie on any of the bidentate or tridentate ligands.
The various dyes emit or absorb over the range of 380-1300 nm, the exact range being dependent on the particular dye.
The complex has a Stokes shift of at least 50 nm, such as at least 100 nm, for example at least 150 nm.
The complex may be luminescent.
The solubility of the dye may be controlled through one or more of: the selection of the ligands, coupling the dye to sugars, changing the charge compensating counter ion of the dye or through the type of protein/peptide attached to the dye.
Dyes are long-lived, typically with lifetimes, in deaerated media exceeding 200 ns, for example exceeding 1 μs. Typically lifetimes may be between 50 and 400 ns in aerated media or between 350 and 500 ns in deaerated media whereas conventional organic dyes have a lifetime or less than about 10 ns in deaerated and aerated media.
Dyes are designed so that their luminescent intensity, lifetime and resonance Raman signature depend on their environment, e.g., oxygen partial pressure, cell/membrane/extracellular redox potential, pH, metal ion concentration and hydrophobicity. Therefore, both quantitative and qualitative sensitivity to their environment is achieved.
The nature of the ligands can be modified to tune sensitivity and emission wavelength.

### Protein

One or more proteins/peptides may be covalently bound to the linker(s) so as to confer a specific biological function, such as but not restricted to, transport across the cell membrane, localisation within a particular tissue/cell type, localisation within a sub-cellular structure, delivery of a therapeutic agent etc. The biological function of peptides may be combined, for example, a dye conjugate may comprise one peptide for localisation and one peptide for transport. Alternatively, both of these functions may be combined within a single peptide.

Optionally, the dye may be covalently bound to one or more linkers with the protein being covalently bound to a second terminus of the linker.

Peptide sequences may be employed to target localisation of the dye conjugate in specific cell organelles, e.g. Lys-Gly-Gly-Pro-Lys-Lys-Lys-Arg-Lys-Val. (SEQ ID No 1) used to target the mitochondria. The technology of the invention can be applied to any peptide or polypeptide sequences for example see Edwards et al and Kieran et al. Examples of peptides that can be conjugated to the dyes described herein include, but are not restricted to, sequences from Cell Penetrating Peptides (CPPs), such as poly-arginine (eg. Arg-Arg-Arg-Arg-Arg-Arg-Arg-Arg SEQ ID No. 2), HIV-TAT (eg. HIV-TAT48-60: Tyr-Gly-Arg-Lys-Lys-Arg-Arg-Gln-Arg-Arg-Arg SEQ ID No. 4), Kaposi-Fibroblast Growth Factor (Ala-Ala-Val-Ala-Leu-Leu-Pro-Ala-Val-Leu-Leu-Ala-Leu-Leu-Ala-Pro-Lys-Lys-Lys SEQ ID No. 5), Nielsen (Lys-Phe-Phe-Lys-Phe-Phe-Lys-Phe-Phe-Lys SEQ ID No. 6) and Antennapedia (Arg-Gln-Ile-Lys-Ile-Trp-Phe-Gln-Asn-Arg-Arg-Met-Lys-Trp-Lys-Lys SEQ ID No. 7); hybrid sequences of CPPs and fusogenic peptides, for example TAT-HA (Arg-Arg-Arg-Gln-Arg-Arg-Lys-Lys-Arg-Gly-Gly-Asp-Ile-Met-Gly-Glu-Trp-Gly-Asn-Glu-Ile-Phe-Gly-Ala-Ile-Ala-Gly-Phe-Leu-Gly SEQ ID No. 8); Nuclear Localization Signal (NLS) peptides, such as NF-κB (Val-Gln-Arg-Lys-Arg-Gln-Lys-Leu-Met-Pro SEQ ID No. 9) and Oct-6 (Gly-Arg-Lys-Arg-Lys-Lys-Arg-Thr SEQ ID No. 10); sequences from Host Defence Peptides (eg. magainin-2: Gly-Ile-Gly-Lys-Phe-Leu-His-Ser-Ala-Lys-Lys-Phe-Gly-Lys-Ala-Phe-Val-Gly-Glu-Ile-Met-Asn-Ser SEQ ID No. 11; buforin-2: Thr-Arg-Ser-Ser-Arg-Ala-Gly-Leu-Gln-Phe-Pro-Val-Gly-Arg-Val-His-Arg-Leu-Leu-Arg-Lys SEQ ID No. 12; pyrrhocoricin: Val-Asp-Lys-Gly-Ser-Tyr-Leu-Pro-Arg-Pro-Thr-Pro-Pro-Arg-Pro-Ile-Tyr-Asn-Arg-Asn SEQ ID No. 13); homing sequences such as c(RGDfK SEQ ID No. 14 in which f is D-phenylalanine); other peptide ligands of integrins; biologically active peptides with membrane translocating properties, or otherwise fused to CPP sequences (eg. BH3 domain of Bid fused to OctaArg: (Arg)₈-Glu-Asp-Ile-Ile-Arg-Asn-Ile-Ala-Arg-His-Leu-Ala-Gln-Val-Gly-Asp-Ser-Met-Asp-Arg SEQ ID No. 15; BH4 domain of antiapoptotic Bcl-X_{L} fused to the protein transduction domain of HIV TAT: Arg-Lys-Lys-Arg-Arg-Gln-Arg-Arg-Arg-Ser-Asn-Arg-Glu-Leu-Val-Val-Asp-Phe-Leu-Ser-Tyr-Lys-Leu-Ser-Gln-Lys-Gly-Tyr-Ser SEQ ID No. 16); or modified as lipo-peptides, (eg. CD226: Pal-Arg-Arg-Glu-Arg-Arg-Asp-Leu-Phe-Thr-Glu SEQ ID No. 17; OCLN: Pal-Lys-Thr-Arg-Arg-Lys-Met-Asp-Arg-Tyr-Asp SEQ ID No. 18; ITGA2B: Pal-Gly-Phe-Phe-Lys-Arg-Asn-Arg-Pro-Pro-Leu SEQ ID No. 19, where Pal=palmitic acid) respectively; their peptide isosteres and their stereo-isomers, including retro-, inverso-, retro-inverso- and partially modified retro-inverso-peptides.

Any suitable peptide can be conjugated to the dye complex such as peptides described in: Org Biomol Chem. 2008 Jul. 7; 6(13):2242-55, Nature Medicine, 10(3):310-315 (2004), Chemistry & Biology, (8):943-948 (2002), Biochem. J. (2006) 399, 1-7, Expert Opinion on Pharmacotherapy, 653-663, 7(6), 2006, Expert Opinion on Investigational Drugs, 933-946, 15(8), 2006, Anti-Cancer Agents in Medicinal Chemistry, 2007, 7, 552-558, Arch Immunol Ther Exp, 2005, 53, 47-60, and Nature Chemical Biology, 2007, 3 (2), 108-112, the entire contents of which are incorporated herein by reference.

The presence of the protein-dye conjugate can be qualitatively or quantitatively detected using optical microscopy including intensity or lifetime based fluorescence, resonance Raman, absorbance, or hybrid techniques, for example:
Single Mode and Hybrid Cell and Tissue Based Imaging Systems, for example combined luminescence and resonance Raman intensity based cell imaging. The small size of the labels allows for more favourable delivery to cells, e.g., tumour cells, compared to higher molecular weight imaging agents
Disease Diagnosis and Mechanism, for example optical methodologies for the detection, diagnosis and monitoring of disease or disease related processes such as early cancer detection, imaging and therapy monitoring either in vivo (endoscopy) or ex vivo. Other applications include probing the role of platelet adhesion in cardiovascular disease. These approaches would include protein-protein interaction and DNA-protein interactions
Theranostics, for example targeted imaging probes for evaluation of patient response to therapy as well as using the peptide-label conjugate as a therapeutic, e.g., photodynamic therapy or engineering of the peptide-label conjugate to create pro-drugs that can be cleaved by specific proteases

[Ru^{II}(L-L)₂(PicH₂)]²⁺ complexes, wherein (L-L)₂ is a bidentate or tridentate heterocyclic ligand containing O and/or N, have appealing luminescent properties, such as long-lived luminescence (ca. 1 μs), and the emission features of these complexes are pH dependent. The pendant carboxylic function of these complexes has excellent reactivity, allowing efficient coupling reactions with amine- and alcohol functionalities. We have synthesized a number of luminescent, ruthenium tagged polypeptides, resulting from the combination of complexes (for example: Ru^{II}(L-L)₂(PicH₂)²⁺ with oligomers of eight (Ru-Ahx-R₈) and five (Ru-Ahx-R₅) arginine units, wherein Ahx is a hexamethylene spacer inserted between the ruthenium luminophore and the polypeptide to minimise interactions between the luminophore and polypeptide that could lead to a quenching of the emission properties of the complex.

Depending on their structure, the dye conjugates are capable of active or passive transport across the cell membrane without causing damage to the cell and emitting visible light from within the cell. Alternatively, the conjugates can be engineered for labelling extra-cellular structures. For example, [Ru(bpy)₂(piCH₂)]²⁺ attached through a hexyl spacer to KVGFFKR-NH₂ (SEQ ID No. 20) can bind to integrin proteins in the cell membrane. Luminescent dye conjugates that are capable of passive cell delivery may be used as molecular probes for example in fluorescence cellular imaging, cell biology, molecular biology, microbiology, and flow cytometry applications. The dye molecules may also be used as environmentally sensitive probes for fluorescence imaging, or luminescent probes for specific targeting to sub-cellular structures and organelles for example by changing the peptide identity and for environmental probing of these structures and organelles such as by fluorescence lifetime imaging (FLIM) to demonstrate for example oxygen and pH sensitivity, or for resonance Raman mapping to demonstrate pH sensitivity. Dyes with redox capabilities within the range of cellular function for example mitochondrial membrane potentials can be used with luminescence imaging, whereby luminescence of the dye is switched off or the lifetime of the dye is dramatically reduced when the dye is oxidised. Alternatively, resonance Raman can be used to image the distribution of redox states of a dye in response to potentials across the cell where changes to the absorbance (and therefore resonance condition) report directly on the redox state of the dye. This is reflected in changes to the resonance Raman spectrum as described in Keyes et al (2002), the entire contents of which is incorporated herein by reference, demonstrates the redox states of ruthenium-ligand complexes.

Dye molecules of the invention can be considered as conjugates or complexes of the general formula:

[M(L¹)ₐ(L²)_{b}(L³)_{c}]-X_{d}-pepₑ

wherein: M is a metal selected from osmium, ruthenium, rhodium, rhenium or copper;
L¹, L², L³ are bidentate or tridentate heterocyclic ligands containing O and/or N and may be the same or different for example, but not restricted to; 2,2-bipyridyl (bpy), 2,2-biquinoline (biq), 4,7-diphenyl-1,10-phenathroline (dpp), 2,3-bis(2-pyridyl)pyrazine (dppz) and 2-(4-carboxyphenyl)imidazo[4,5-f][1,10] phenanthroline (piCH₂);
a, b, c are integers between 1 and 3 and may be the same or different and wherein the sum of a+b+c is 2 or 3;
X is a functional group for directly or indirectly covalently binding to Pep wherein the functional group for directly covalently binding to Pep is selected from: amine, carboxylic acid, thiol or azide reactive functionalites such as, carboxylate, amine, iodoacetamides, maleimides, active esters such as succinimidyl esters and hydroxybenzotriazole esters, alkyl halides, acid halides, isothiocyanates, azide or alkynes;
Pep is a peptide/polypeptide/protein containing at least 3 amino acids;
d and e are integers between 1 and 3 and are the same and wherein the integers for d and e are equal to or less than the sum of a+b+c; and
wherein there is optionally a linker between X and Pep

The conjugate may comprise a linker molecule between the peptide and ligand. The linker may be an α-amino-acid or a higher homologue such as an ε-amino-acid to a ι-amino-acid. In the case of peptides and/or ligands that are poorly soluble in aqueous media, a polymer of ethylene glycol (PEG) may be used as a linker to improve the water solubility of the dye conjugate.

We have synthesised a series of luminescent bio-probes for lifetime and intensity based luminescent imaging. Different organic fluoropheres (e.g. FITC, Rhodamine derivatives and the like) (Zhao et al: Kolodziejczyk et al; Morris et al) have been covalently linked to, antibodies, and proteins. However, in the case of organic probes, these materials exhibit short emission lifetimes and strong bleaching with irradiation time, preventing studies on longer time scales. The lifetime of the luminescence of the dye conjugates described herein depends on the nature of the ligand ranges and is substantially longer than luminescence lifetimes of common commercial organic probes thus allowing auto fluorescence from the biomaterial itself to be eliminated, for example by accumulating the emitted photons after a delay time excitation.

An exemplary example of a conjugate is a ruthenium polypyridine compound for example [Ru(dppz)₂(PicH₂)]²⁺ of the formula:

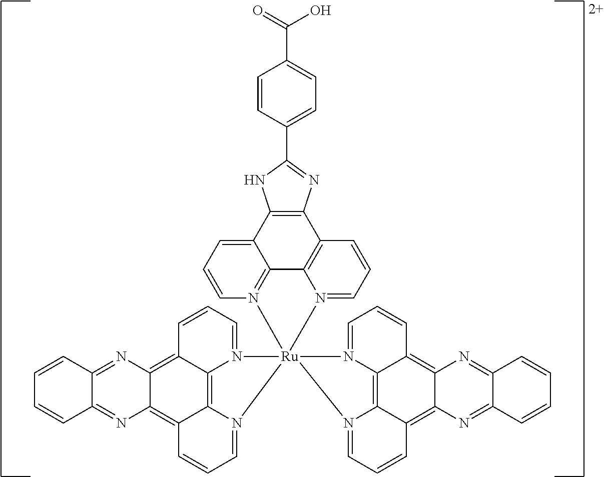

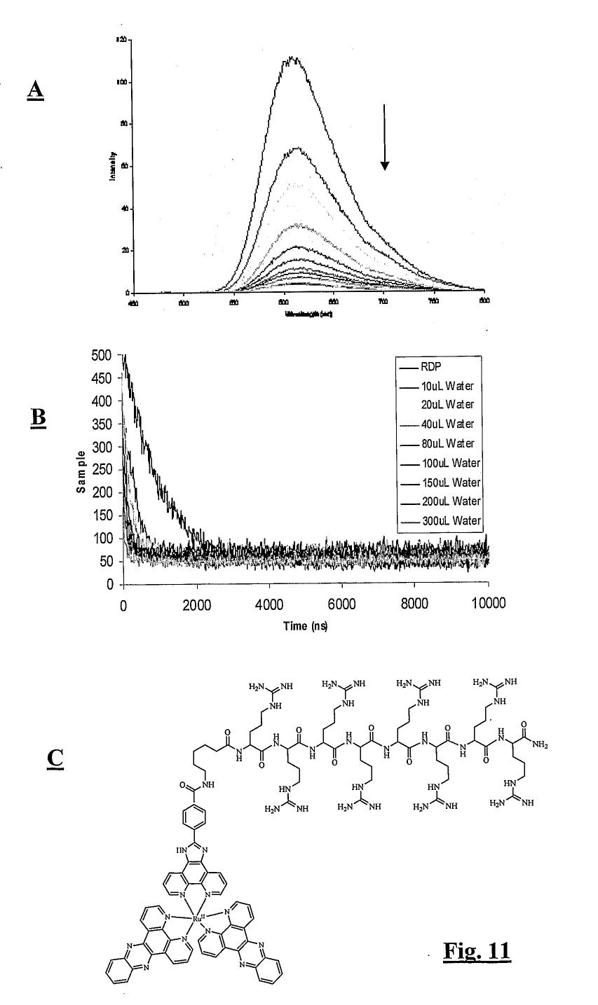

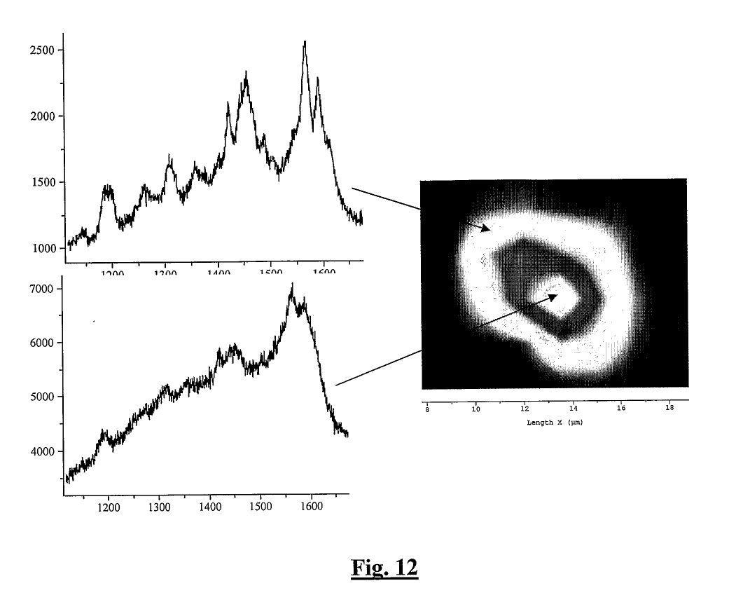

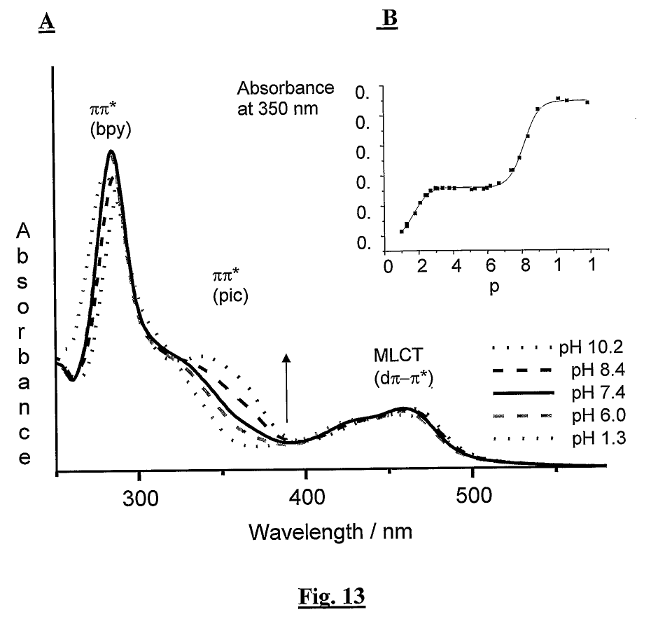

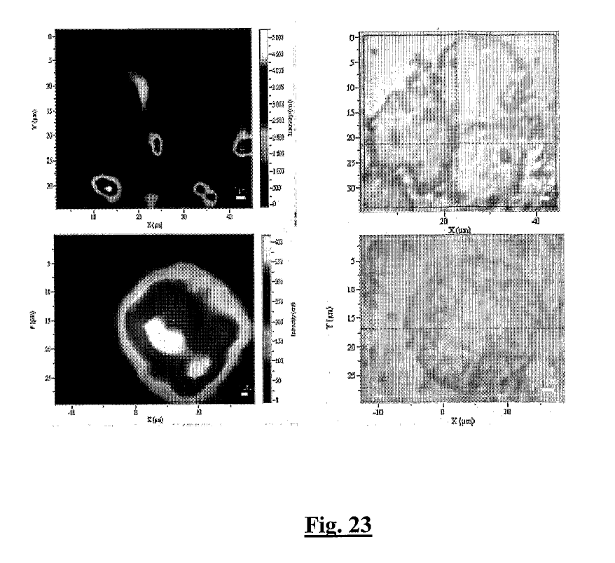

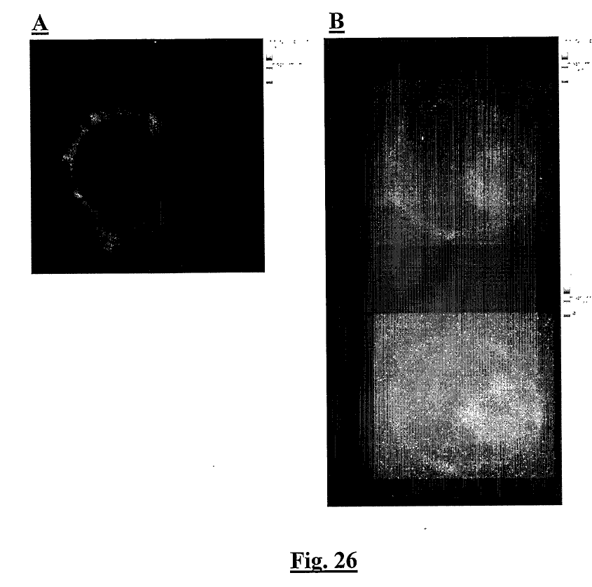

The [Ru(dppz)₂(PicH₂)]²⁺, is useful as a membrane probe as the complex is water sensitive and only emits a signal in a hydrophobic environment. Its R₈ conjugate is shown in FIG. 11 C along with its luminescence intensity FIG. 11 A and lifetime dependence FIG. 11 B, on H₂O concentration. Using emission imaging, only emissions from membrane regions of a cell can be detected, FIG. 26, however the general distribution of the complex (i.e. the overall distribution within both membrane and non-membrane regions of a cell) can be mapped by resonance Raman. regardless of whether the dye emits or not. For example, FIG. 25 shows the resonance Raman spectroscopy of [Ru(dppz)₂(PicH₂)]²⁺ exciting at 458 nm and FIG. 23 shows resonance Raman map of [Ru(dppz)₂(PicHR₈)]²⁺ incubated with myeloma cells.

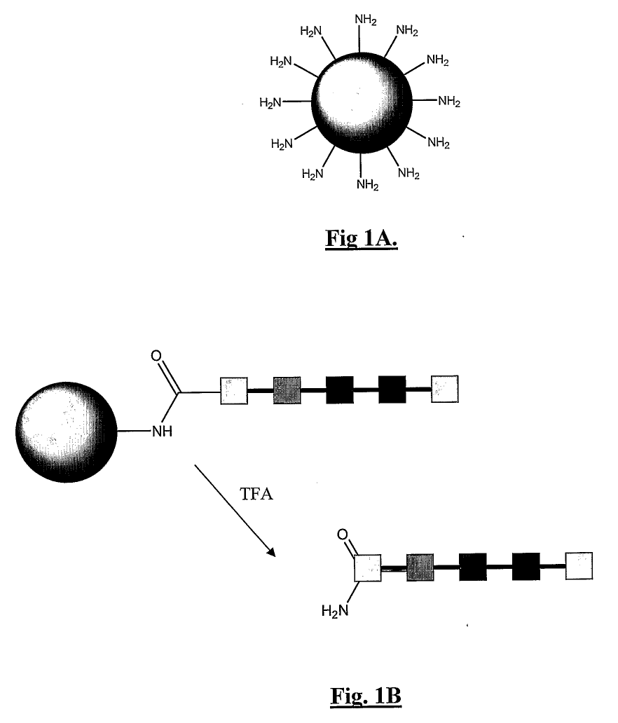

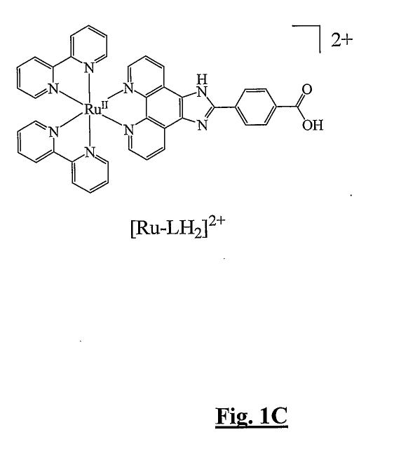

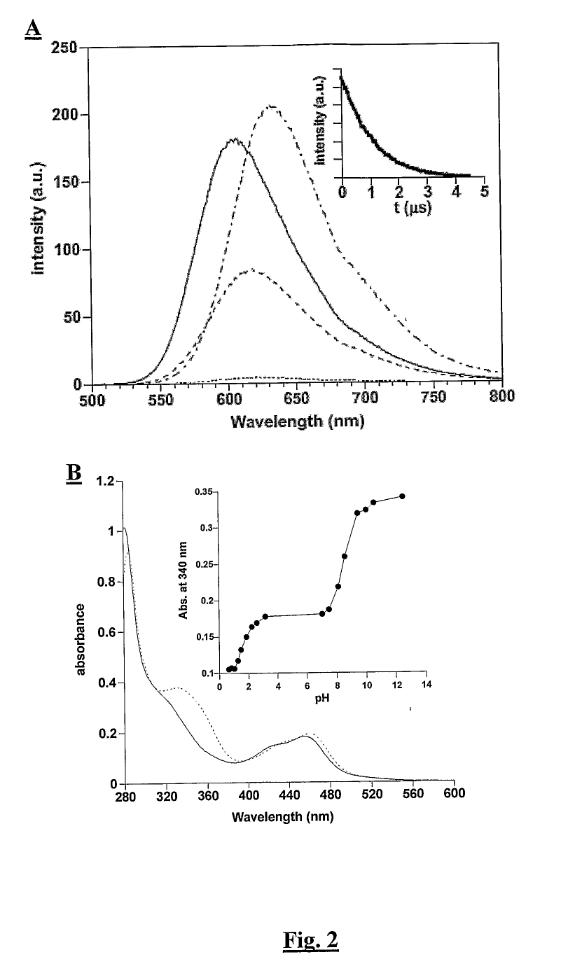

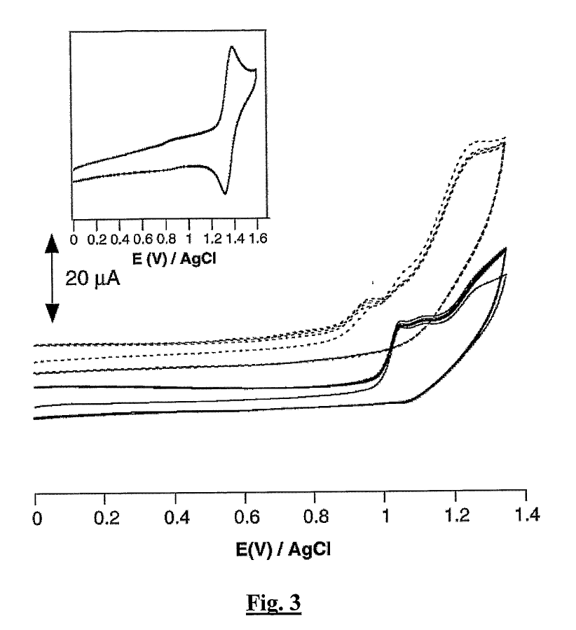

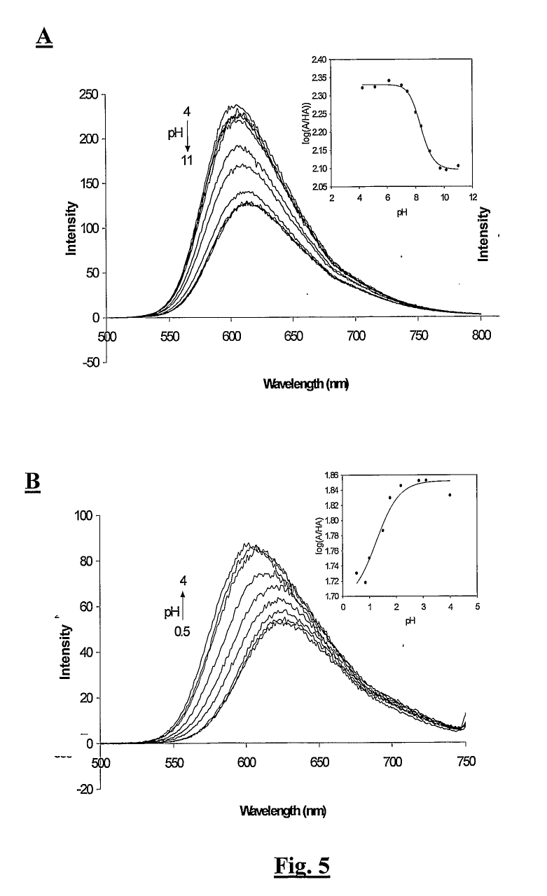

Another example of a dye conjugate is [Ru(bpy)₂(PicH₂)]²⁺, FIG. 1c The [Ru(bpy)₂(PicH₂)]²⁺ complex is pH, FIGS. 2, 3, 4 and 5 and oxygen sensitive and can therefore be used for imaging the pH and/or oxygen concentration within a cell.

A peptide such as octa arginine (R₈, SEQ ID NO. 2) can be covalently attached to ruthenium polypyridine compounds to form Ru-Ahx-R₈ in which Ahx is a linker such as 6-amino hexanoic acid. Ru-Ahx-R₈ can be synthesised by covalent linkage of an octa-arginine oligopeptide to a ruthenium polypyridine luminophore, via an aliphatic hexamethylene spacer. Oligoarginine polypeptides are a well-documented class of biocompatible entities, proven to be capable of penetrate the cells without damaging their membrane. In some instances, they can bring with them covalently attached drug/probe (Goun et al).

Ruthenium polypyridine complexes form a well-known family of long-lived, oxygen-sensitive, inorganic luminophores (e.g. Medlycott et al). They have been applied in a range of sensing capacities, but there are few examples of their use in cellular imaging, and no examples of peptide labelled Ru complexes for cellular imaging. We have found that Ruthenium polypyridine compounds can be incorporated into a dye complex of the formula:

[M(L¹)ₐ(L²)_{b}(L³)_{c}]-X_{d}-Pepₑ

wherein as an exemplary example:
M is the metal Ruthenium;
L¹ and L² are the bidentate ligand dppz;
L³ is the ligand PicH₂ which bears carboxylic acid function
X is a functional group linked to the linker 6-amino hexanoic acid;
a, b and c are 1;
Pep is octa arginine (SEQ ID NO. 2); and
d and e are 1.

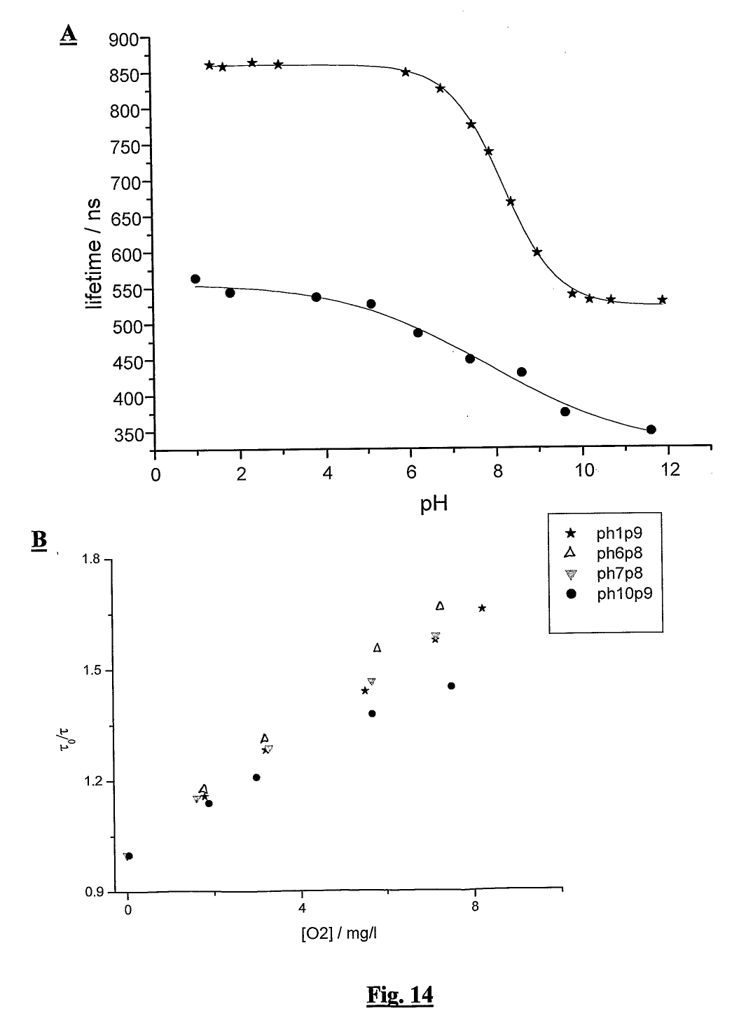

The emission properties of [Ru(bpy)₂PicH-R₈]ⁿ⁺ change in relation to an alteration in pH, FIG. 14 A as the imidazole unit of the ligand becomes protonated and deprotonated. But the system remains strongly emissive even at pH 10, which is above the level of physiological pH. The emission of [Ru(bpy)₂PicH-R₈]ⁿ⁺ is oxygen sensitive. However, the oxygen sensitivity does not depend on pH as shown in FIG. 14 B, as the slope of the Stern-Volmer plot remains constant over a range pH 1 to 10. Therefore the measure of the luminescence lifetime of [Ru(bpy)₂PicH-R₈]ⁿ⁺ may be used to detect intra-cellular oxygen as well as a changes in local concentration of oxygen. For dyes with redox accessible states, changes to redox state will appear as quenching of luminescence and resonance Raman spectroscopy and/or fluorescence imaging can be used to map or image the redox distribution of the dye.

[Ru(bpy)₂PicH-R₈]ⁿ⁺ can be easily synthesised and purified, and is obtained with a good yield. It is a “user-friendly cell tag” with high synthetic yield suitable for many kinds of cellular imaging experiment. The ruthenium centre is resistant to photobleaching it is long-lived and intense, and has absorption and emission characteristics that are compatible with most conventional confocal laser systems. The long lifetime emission of the ruthenium complex makes it quantitatively sensitive to oxygen concentration and the ligands can be readily altered to permit sensitivity to pH, water content and the rigidity of the microenvironment.

The invention will be more clearly understood from the following examples thereof.

### EXPERIMENTAL

### ¹H NMR spectra

¹H NMR spectra were recorded on a Bruker Advance series 400 MHz NMR spectrometer. The NMR titration experiments were performed following a previously published protocol (Charbonnier and Penades). Mass spectra were acquired using a positive ion mode on a Bruker LC/MS Esquire. Electronic absorption spectra were measured on a Shimadzu 3500 UV-VIS/NIR spectrophotometer. Cyclic voltammetry was carried out using a CH Instruments CH602 electrochemical workstation. A conventional three-electrode cell was used, employing glassy carbon as working, Ag/AgCl as reference or Ag/AgNO₃ in acetonitrile, and Pt wire as counter electrodes. Electrochemistry was conducted in water and acetonitrile respectively with 0.1 M phosphate buffer or tetrabutylammonium tetrafluoroborate (TBABF₄) as supporting electrolyte under an N₂ atmosphere. pH titrations were performed in the pH range 0.5-12. The pH was adjusted by adding aqueous solutions of NaOH or HClO₄.

### Resonance Raman Spectroscopy

Resonance Raman spectroscopy was performed on a Horiba Jobin Yvon HR800UV confocal microscope using an Argon ion Laser (458 nm or 514 nm) or a Helium-Neon (HeNe) laser (633 nm) as the exciting wavelength. Ten spectral acquisitions were accumulated and each acquisition was two seconds in length. Steady-state emission spectra were recorded on a Cary Eclipse Fluorescence spectrophotometer, and luminescence lifetimes were obtained using a Picoquant Fluotime 100 TCSPC system exciting at 470 nm and detecting at 600 nm using a narrow band pass dielectric filter. Quantum yields were measured using the comparative method described by Williams et al. In quenching studies, the luminophore/quencher concentrations were corrected for dilution.

### Synthesising Dye-Peptide Conjugates

Peptides were prepared by standard Solid Phase Peptide Synthesis according to the Fmoc-tBu strategy with HBTU/HOBt/DIEA coupling chemistry, in N-methylpyrrolidone (NMP) solvent. Single coupling cycles using a 10-fold excess of Fmoc amino acid derivatives to resin-bound peptide were employed. The side chain protecting groups were Pbf for Arginine, the syntheses were carried out on a 1.0×10⁻⁴ mol scale. Assembly of the amino acid sequence, starting from a Rink Amide MBHA resin and attachment of the N-terminal spacer were carried out on an automated peptide synthesizer (Applied Biosystems 433A).

Typically, 650 mg (1 mmol) of Fmoc-Arg(Pbf)OH were used for each coupling reaction (5 or 8 times) for 140 mg of rink amide resin (loading 0.72 mmol/g). 1 mmol of the spacer (N-fmoc-6-aminohexanoic acid) was used before removal of the resin from the automated synthesizer. The labelled peptides were prepared by attachment of a fluorogenic substrate (Ru(bpy)₂(PicH₂)(ClO₄)₂, 290 mg, 300 μmol) on the N-terminal spacer using PyBOP (300 μmol, 160 mg), HOBt (300 mol, 60 mg), DIEA (80 μL) coupling chemistry. The reaction was performed overnight in a plastic cell, at room temperature, in dark. Peptides were deprotected and cleaved from the synthesis resin using a mixture of 80% trifluoroacetic acid, 5% water, 5% triisopropylsilane, 10% thioanisole at room temperature for 4 h. The peptides were precipitated and washed three times with 10 ml portions of diethyl ether. They were then dried, dissolved in distilled water and lyophilized.

Chromatographic analysis and purification were performed on a BioCAD SPRINT Perfusion Chromatography Workstation (PerSeptive Biosystems) using Gemini columns (5 Å, C18, 4.6 mmd/250 mL (analytic) 100 mmd/250 mL (semi-preparative), Phenomenex). (A mobile phase: 0.1% TFA in water; B mobile phase: 0.1% TFA in acetonitrile). Gradient: 2 to 60% B in 18 column volumes; flow rate: 4 ml/mn; single wavelength detection at 214 nm.

The peptides were characterised by Matrix Assisted Laser Desorption Ionisation—Time Of Flight—Mass Spectrometry (α-cyano-4-hydroxy-cinnamic acid matrix).

### Environmental Sensitivity Towards pH and Oxygen

The pH was adjusted with H₂SO₄ (Sigma-Aldrich) and KOH (Sigma-Aldrich) and measured with a pH meter. UV/vis absorption spectra during the pH titration experiment were corrected for change in volume.

Different oxygen concentrations were adjusted by streaming various O₂—N₂-mixtures through the solution until equilibrium was reached. The oxygen concentration was measured with an optical O₂ electrode (Visiferm DO120, Hamilton). The fluorescence lifetime of the equilibrated dye solutions was measured in a closed-system flow cell.

For the fluorescence and fluorescence lifetime experiments 100 μl aliquots were used and incubated with the 3 μl of the Ru-octopeptide (1.2 mM), resulting in a final dye concentration of 3.5×10⁻⁵ M. If only resonance Raman experiments were performed, the cells were incubated in the medium with dye for 15 minutes prior to the washing step.

### Myeloma Cells

Sp2/0-Ag 14 myeloma cells (ATCC number CRL-1581™) were obtained from the ATCC Cell Biology Collection (United Kingdom). Cells were grown in Dulbecco's modified Eagle's medium (Sigma-Aldrich) supplemented with 10% foetal calf serum (Gibco Invitrogen) and 1% L-glutamine (Gibco Invitrogen) at 37° C. and 5% CO₂. Cells were harvested after growing for 2 days. The viability was ensured by testing them in a trypan blue assay. In a usual sample less than 1% of the cells were dead. The growth medium was removed by centrifuging the cells at 2000 rpm for 2 min in an Eppendorff centrifuge, washed twice and resuspended in PBS buffer.

Bovine aortic endothelium cells were supplied by Dr PM Cummins and cultured as described in Colgon et al.

Human Blood Platelets were supplied by Niamh Moran and Dermot Kenny and prepared as described Moran et al.

### Localization of the Ru Dye Inside the Cell/Counterstain Experiments

To identify where the Ru dye [Ru(bpy)₂(PIC-arg₈)]²⁺ is localizing inside the cell, cells were counterstained with two commercial dyes. Sytox green (Invitrogen) which localizes inside the nucleus; and DiOC6(3) (3,3′-dihexyloxacarbocyanine iodide, Invitrogen) which selectively stains mitochondria and at higher concentration other internal membranes, such as the endoplasmatic reticulum, of living cells. Both dyes can be excited with the 458 nm line of an argon ion laser used for excitation of the [Ru(bpy)₂(pic-R₈)]ⁿ⁺ dye, however, their fluorescence is in the green (around 500 nm), and therefore could be easily spectrally separated from the fluorescence of the Ru complex. However, Sytox green only stains only cells with a compromised membrane. Therefore, in the first hours of incubation with the Ru dye, Sytox green was used to prove that the cells are still intact. The size and shape of the nucleus of myeloma cells was investigated using cells with permeabilized membranes (using Triton 1% v/v).

### Luminescence and Luminescence Lifetime Imaging

For the fluorescence and fluorescence lifetime experiments 100 μl aliquots of washed myeloma cells were resuspended in PBS buffer (pH 7.4) and incubated with the 3 μl of the Ru-octopeptide (1.2 mM), resulting in a final dye concentration of 3.5×10⁻⁵ M.

The luminescence images of myeloma cells were recorded with a Zeiss LSM510 Meta confocal microscope, using a 63× oil immersion objective (NA 1.4). The 458 nm line of an argon ion laser was used for excitation. OD filters were used to reduce the laser to 0.1% transmission in order to avoid possible photobleaching. The luminescence from the Ru complexes was collected using a longpass filter at 560 nm. When Sytox green and/or DiOC6(3) were localised in the same cell, the luminescence from the Ru-dye was collected behind a 615 nm longpass filter, while the Sytox green and DiOC6(3) fluorescence was collected with a 465-510 nm bandpass filter in another channel, or by using the meta option of the instrument.

The luminescence lifetime images were recorded with a Picoquant lifetimes upgrade system on the Zeiss confocal microscope. A 405 nm pulsed laser with a repetition rate of 500 000 Hz (external trigger) was used to excite the sample. The fluorescence light above 530 nm was collected on a SPAD detector into 4000 channels.

In the myeloma mapping experiments, maximum counts were reached after 7 minutes of acquisition. For calculating a false colour lifetime image 2×2 pixels were binned and the luminescence decay fitted with a monoexponential curve using the Picoquant software. For an estimate of the lifetimes in different regions the photon counts of those regions were added and fitted with a monoexponentioal decay.

### Data Analysis

UV/vis absorption titration curves were fitted with a modified Henderson-Hasselbalch equation which was obtained using Beer-Lambert's law to express the concentration, FIG. 2(b) For fitting the fluorescence titration curve it was assumed that the average lifetime goes linear with the ratio of the two species in equilibrium at the certain pH.

To evaluate the pH dependence of the resonance Raman data peak ratios were calculated with Labspec5 software. A baseline was drawn along the base of the peak and the area was calculated. Regions for the peaks were 1700 to 1400 cm⁻¹. The ratio of the peaks for the different protonation states was 5 replica measurements were recorded for each pH and averaged.

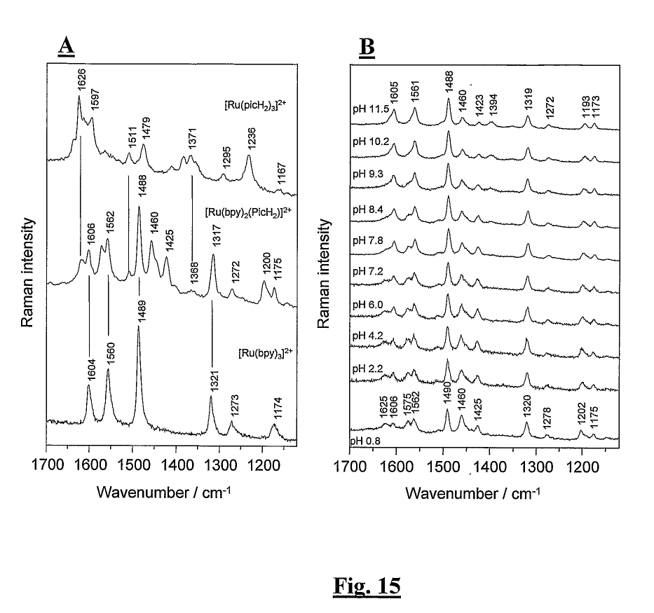

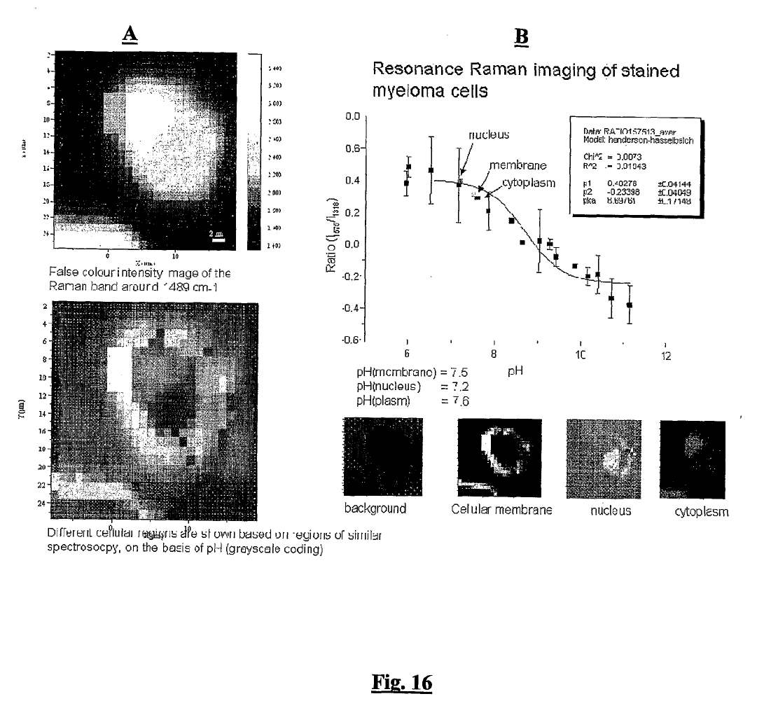

The resonance Raman maps of the stained myeloma cells were analyzed using the modelling option implemented in the LabSpec software. A model with 4 components was creating, grouping together similar spectra in one component (background, where there is no dye/no cell, outer cell (membrane), cytoplasm and nucleus). From those average spectra from each component the peak ratio of the pH sensitive with the pH insensitive peaks was determined and used to estimate the pH inside the cells using the calibration plot created with the pure dye FIGS. 15 and 16.

### Example 1

### Synthesis of the Metal-Ligand Complex [Ru(bpy)₂PicH₂]ClO₄

Ruthenium trischloride was purchased from Aldrich, N-methyl morpholine, ammonium hexafluorophosphate, 2,2′-bipyridine (bpy), 1,10-phenanthroline and 4-carboxybenzaldehyde were purchased from Aldrich Chemical Company. 1,10-phenanthroline-5,6-dione (Bodige and Mac Donnell), [Ru(bpy)₂Cl₂] (Sullivan and Meyer), 2,4-dimethoxy-1,3,5-triazinemethylmorpholinium chloride (DMTMM) (Kunishima et al) were synthesised as described in the literature.

(4-carboxyphenypimidazo[4,5-f][1,10] phenanthroline (LH₂): 0.21 g of phendione (1 mmol), 0.18 g of 4-carboxybenzaldehyde (1.2 mmol) and 1.54 g of ammonium acetate (20 mol) were refluxed in glacial acetic acid for 3 hours. The yellow solution was then allowed to cool at room temperature. A yellow solid started to precipitate and addition of water afforded more powder. All solid phases were gathered and washed thoroughly with water, methanol and ether. The yield: 0.25 g (73%) and ¹H-NMR (400 MHz, d⁶-DMSO): 14.0 (s, 1H), 13.15 (s, broad, 1H), 9.05 (m, 2H), 8.95 (m, 2H), 8.40 (d, 2H), 8.18 (d, 2H), 7.85 (m, 2H).

The final product had the following structure:

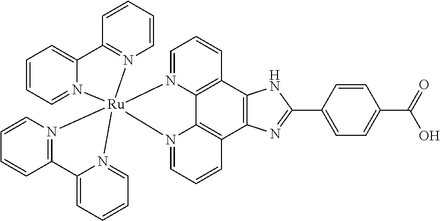

### Example 2

### Synthesis of the Metal-Ligand Complex [Ru(bpy)₂(PicH₂)](PF₆)₂ 0.05 g of

[Ru(bpy)₂Cl₂] (96 μmol) and 0.033 g of PicH₂ (1 equivalent) were refluxed in ethanol for 16 hours, in the dark. The red-orange solution was then evaporated under reduced pressure. The red crude material was then dissolved in the minimum amount of methanol and unreacted ligand was removed by filtration. The filtrate was evaporated and the dark red powder obtained was thoroughly washed with dichloromethane; a light orange powder of [Ru-LH₂]²⁺ was obtained upon addition of aliquots of a concentrated aqueous solution of ammonium hexafluorophosphate. The yield: 0.085 g (85%), the ¹H-NMR (400 MHz, d⁶-DMSO): 9.03 (d, 2H), 8.84 (d, 2H), 8.81 (d, 2H), 8.41 (d, 2H), 8.20 (t, 2H), 8.05 (m, 4H), 7.85 (m, 6H), 7.58 (m, 4H), 7.35 (t, 2H). ESI-MS: m/z=377.0 (M²⁺/2).

### Example 3

### Synthesis of the Metal-Ligand Complex [Ru(dppz)₂(PicH₂)]ClO₄

1.63 mmol of both Ru(dppz)₂Cl₂ (1.12 g) and PIC ligand (0.55 g) were stirred and placed under reflux in ethylene glycol overnight, during which the solution turned dark orange. The solution was vacuum-filtered on a glass frit and washed thoroughly with deionised water. The dark brown powder was dissolved with 50/50 dichloromethane/methanol and the filtrate was then rotary evaporated to remove the solvent and a suspension of the complex was formed in diethyl ether and was vacuum filtered again. The dark orange powder was washed with deionised water and dried with diethyl ether. Yield: 72.2% (1.30 g) MW: 1105 g/mol. ESI-MS: M²⁺1005.6 m/z, M²⁺/2 503.5 m/z

The final product had the following structure:

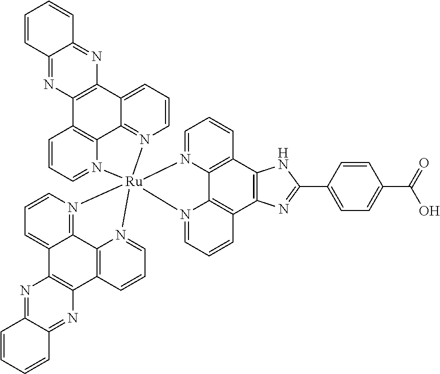

### Example 4

### Synthesis of the Metal-Ligand Complex [Ru(dpp)₂(PIC)]PF₆

0.3 mmol of both Ru(dpp)₂Cl₂ (0.25 g) and PIC ligand (0.1 g) were refluxed in 1:1 ethanol:water overnight, during which the solution turned a dark orange colour. Aqueous lithium perchlorate was added to precipitate the complex. The solution was vacuum filtered in a glass fritz and washed with water and dried with ether. % Yield: 52% (0.19 g). ESI-MS: M²⁺1105.6 m/z, M²⁺/2 553.5 m/z

The final product had the following structure:

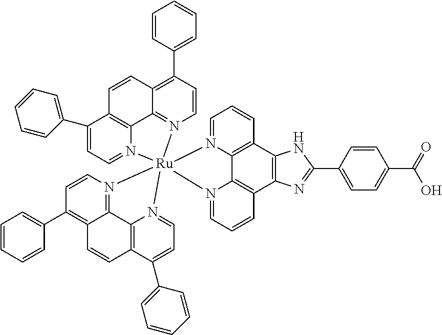

Whilst ruthenium was used as the metal in the metal-ligand complex of the exemplary examples 1 to 4 above, metal-ligand complexes of osmium, rhodium, rhenium or copper can also be synthesised using these techniques.

### Example 5

### Synthesis of the Dye-Derivatized Peptides

Peptides were synthesised using a well-established solid state supported peptide synthesis developed by Merrifield: starting from resin beads (polystyrene) bearing NH₂ functionalities, the first amino acid is covalently linked to the former using standard procedures. The second amino acid of the desired sequence is then attached, and so on (FIG. 1A). The advantage of solid phase peptide synthesis is that the purification steps are all performed in one go by merely washing the resin with appropriate solvents.

The beads are provided with the “substitution”, i.e. the number of moles of substituted groups by grams of bead, which allows to calculate the relative quantities of amino acid to add to perform the reaction.

When the polypeptide is built, one eliminates the bead with trifluoroacetic acid (TFA). The resulting compound is an amide terminated polypeptide (FIG. 1B).

As an exemplary example, the synthesis of a polyarginine peptide is described.

### Synthesis of the Oligopeptide

The synthesis of the oligopeptide is conducted in an automated synthesizer. In this instance, the oligopeptide is a polyarginine (Rₙ) where n is 5 to 20 arginines and linked together via amide bonds.

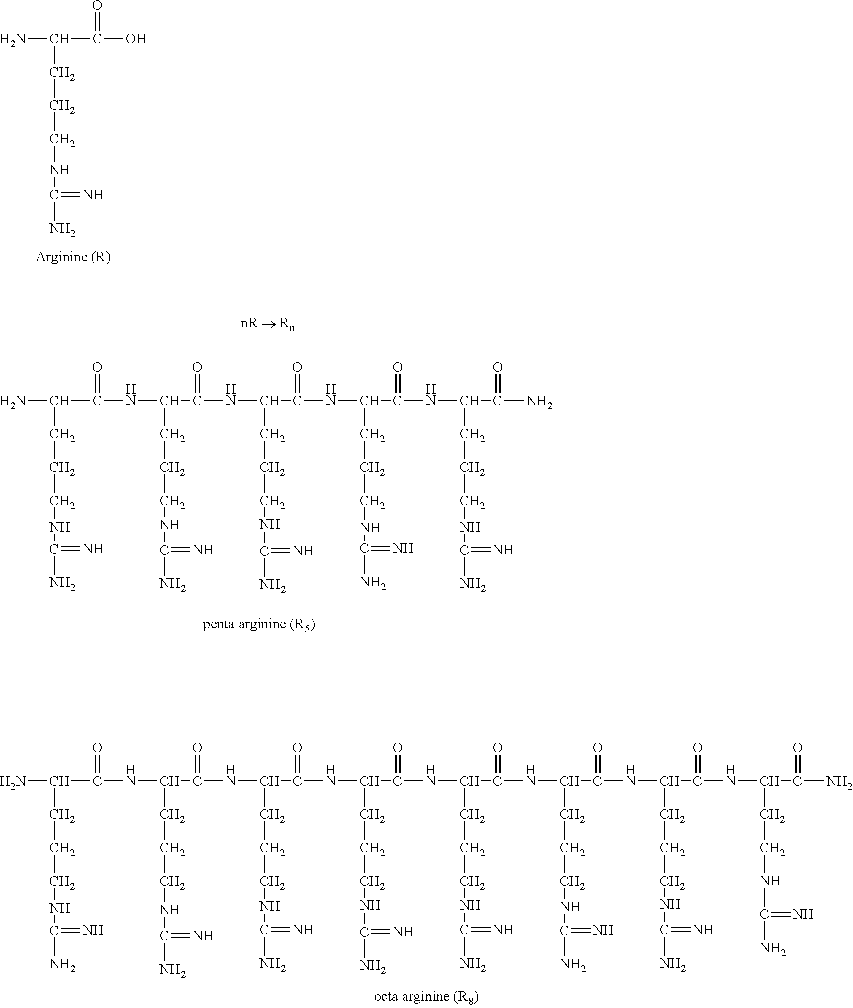

The amino acid can react with itself if no precaution is taken; to avoid undesired cross coupling reactions, we used amine-protected amino acids. The protective group is a FMOC group, removed later by a treatment with a base (piperidine). Moreover, many amino acids possess a side chain that bears reactive functionalities. This is true for arginine, and therefore it is necessary to use a side chain protected arginine (commercially available), such as an arginine with a pentamethyldihydrobenzofurane (“pbf”) group. The pbf group is removed by TFA during cleavage from the resin. The starting protected arginine used in all our syntheses is of the following formula:

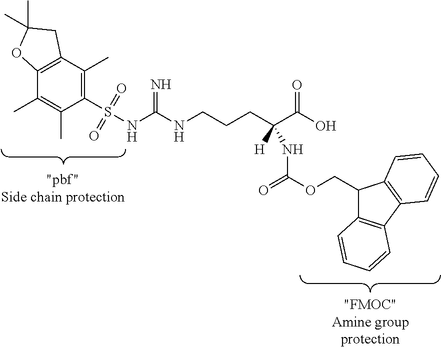

The coupling reaction of one amino acid to a peptide borne by a rink amide resin is achieved by using coupling agents, which enhance the reactivity of the acid function of the amino acid towards the amine function of the peptide. Many coupling agents are used in peptide synthesis, and sometimes two coupling agents are used at the same time to improve yields.

HOBt (1-Hydroxybenzotriazole hydrate) and HBTU (N,N,N′,N′-Tetramethyl-O-(1H-benzotriazol-1-yl)uronium hexafluorophosphate) were used in an automated synthesis apparatus to synthesis octa- and penta-arginine. A mixture of PyBOP and HOBt was employed in the coupling step involving the dye itself.

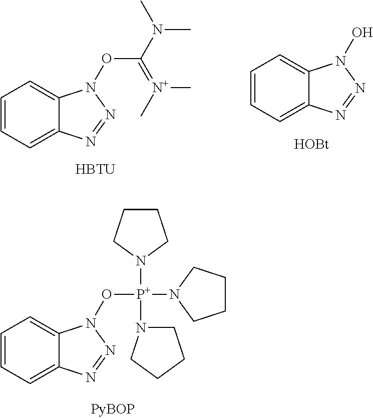

The synthesis protocol is the same for all peptides. The first step is the formation of the first peptide bond onto the amine functionalised resin. This step is done using the following proportions: 1 resin, 10 amino acids, 10 HBTU, 10 HOBt and 20 diisopropylethylamine (DIEA, base). The quantity of resin is roughly 100 μmol.

These steps are then repeated as many times as necessary, namely 5 times for pentaarginine (R₅) (SEQ ID No 3) and 8 times for octaarginine (R₈) (SEQ ID No 2). Each step comprises the formation of the peptide bond, and the removal of the FMOC protecting group, and takes one hour. An additional linker, 6 methylene groups stemming from the coupling of 6-aminohexanoic acid with R₅ or R₈, was linked to the last arginine; the purpose of this step is to prevent any undesired interaction between the peptide and the dye that will be eventually attached.

Insertion of a Linker onto the Oligopeptide Chain (Optional)

A linker was used to avoid undesired interactions between the peptide itself and the molecular entity (here a dye), which must be attached in the next step. The linker is a hexamethylene chain, produced by conjugation of 6-aminohexanoic acid (symbol: Ahx) with the oligopeptides R₅ (SEQ ID No 3) or R₈ (SEQ ID No 2). The remaining amino group of the linker confers a “peptide-like reactivity” to the resulting macromolecule, onto which the dye can then be docked.

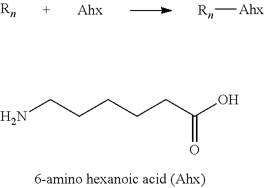

The presence of the linker/spacer is not compulsory but has the advantage that it confers reproducible and predictable reactivity of the oligopeptide towards the dye. Longer linkers up to C₁₀ may also be used.

If the peptide is to be directly linked to the ligand of the dye complex, this step can be omitted. The peptide may be directly linked to the ligand using standard techniques known to a person skilled in the art such as those described in Biochemistry (2006) 45: 12295-12302, the entire contents of which is incorporated herein by reference.

### Conjugation of the Metal-Ligand Dye and Peptide

The conjugation of the Ru(bpy)₂(piCH₂)(ClO₄)₂ dye (Ru) to the oligopeptide-linker species:

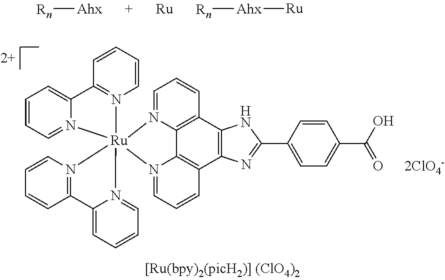

The general formula of this kind of complex is Ru(L¹)ₐ(L²)_{b}(L³)_{c} where L¹ and L² are bpy, or 2,2′-bipyridyl, or the same or different bidentate ligands listed below. Their role is to complete the coordination sphere of the metal, and they may be used to tune the optical properties of the complex.

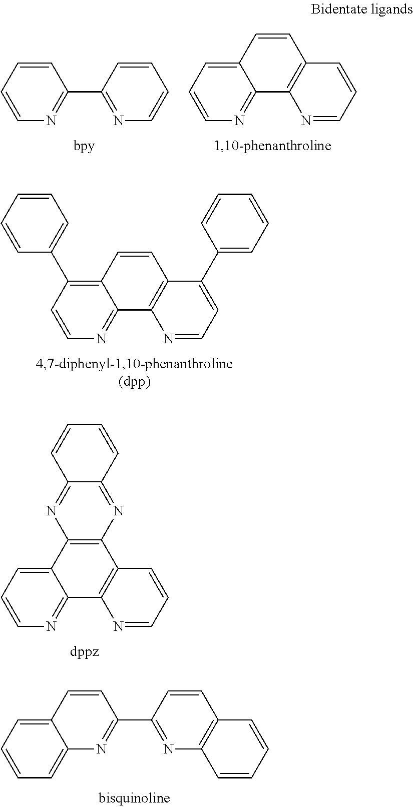

In this example L³ is PicH₂, a ligand bearing a carboxylic acid function, which can be conjugated to the amine function of a peptide and more generally of an amino acid.

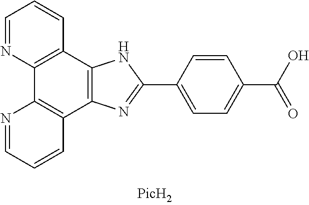

Alternatively, L³ may be selected from one or more of the following carboxyl bearing ligands:

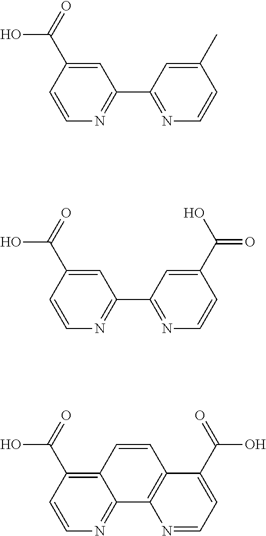

Complexes of the type Ru(L¹)ₐ(L²)_{b}(L³)_{c} (ruthenium-polypyridyl complexes) are reported extensively in the literature for their strong and long-lived luminescence around 600 nm. Other metal ions can be used instead of ruthenium(II) for example osmium(II), rhenium(I), rhodium or copper (II).

To conjugate the peptide to a ligand of the dye complex, the treated resin (off white) was removed and reacted separately with the dye and a new cocktail of coupling agents, as follows: 100 μmol of the resin is dispersed in DMF, while a solution of 300 μmol of the dye, Ru(bpy)₂(piCH₂)(ClO₄)₂, 300 μmol of PyBOP, 300 μmol of HOBt in DMF was prepared. The HOBt in DMF was then added to the resin suspension. 80 μl, of DIEA was finally added to the mixture, which was allowed to stir overnight, in dark.

The burgundy resin was then washed with DMF and dichloromethane; once dry, it was treated with the following mixture: 2500 μL of trifluoroacetic acid (TFA), 150 μL of water, 300 μL of thioanisole and 30 μL of triisopropylsilane, for 4 hours.

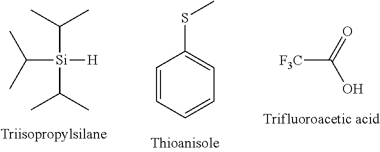

This “cleavage cocktail” is designed to trap all the protective groups like pbf, which are removed by TFA, but could potentially react again with the peptide. The orange solution was filtered, and diethyl ether was then added to crash out an orange powder. The latter was washed with ether, and eventually redissolved in water and lyophilised.

The resulting solid was finally purified by semi-preparative high performance liquid chromatography, using a C18 Gemini column. The collected fractions were characterized by Maldi TOF mass spectrometry, and the ones showing a peak at 2115 g·mol⁻¹ in the case of Ru—R₈ were gathered and stored in the fridge.

The final products obtained had the following structure:

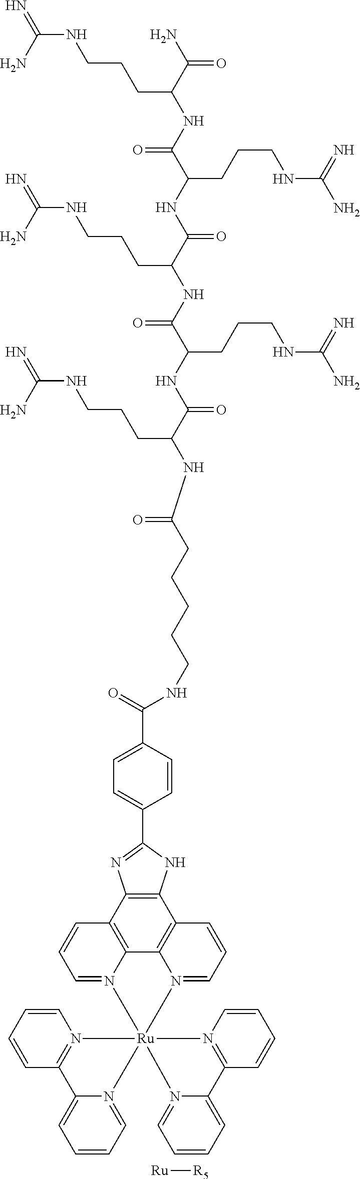

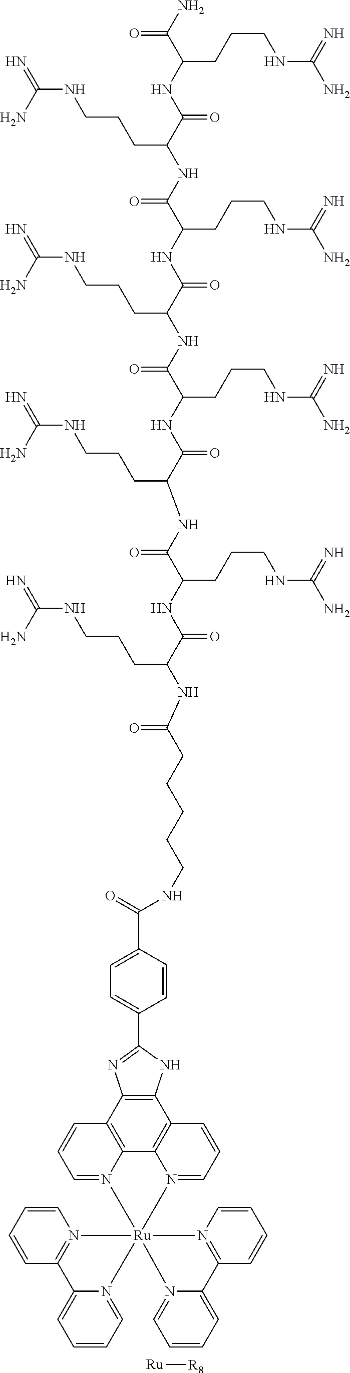

Using this synthesis procedure we have also made the following dye-protein conjugates:

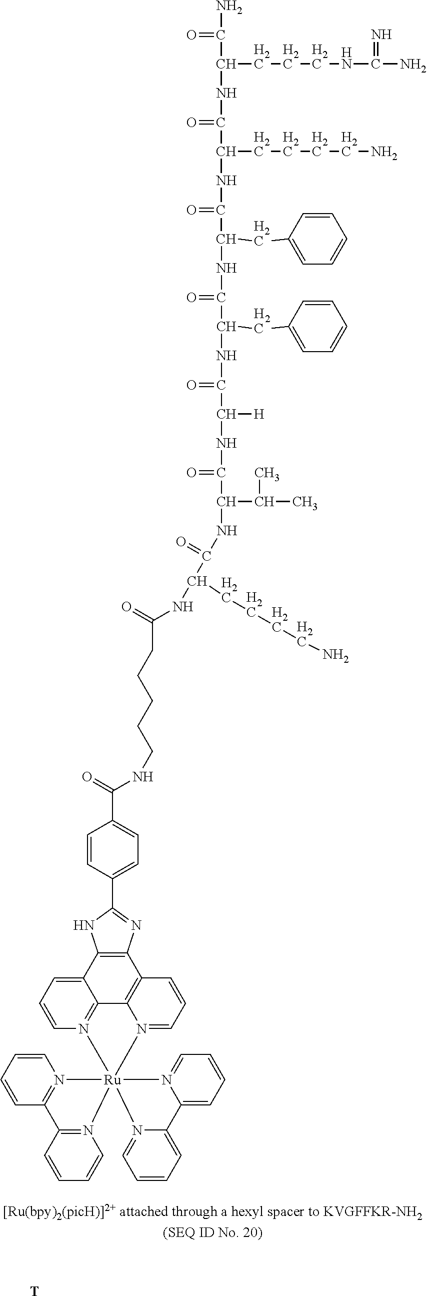

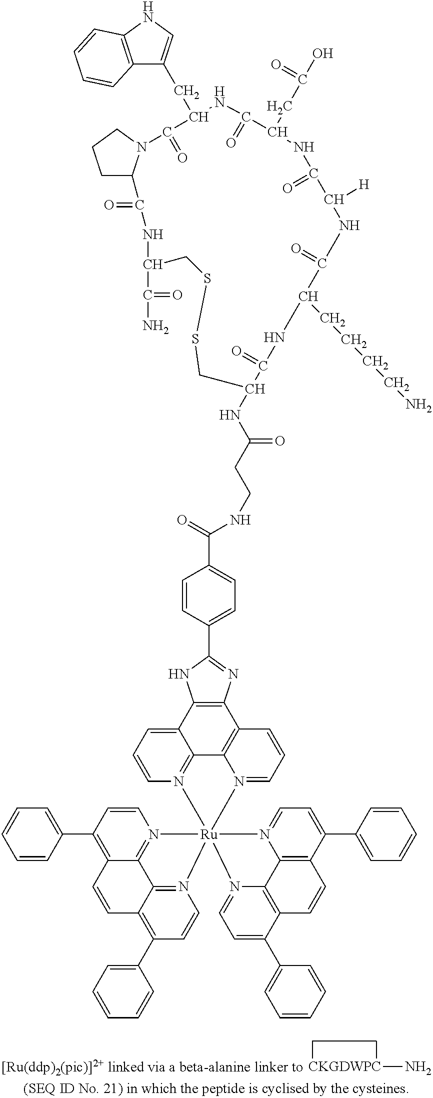

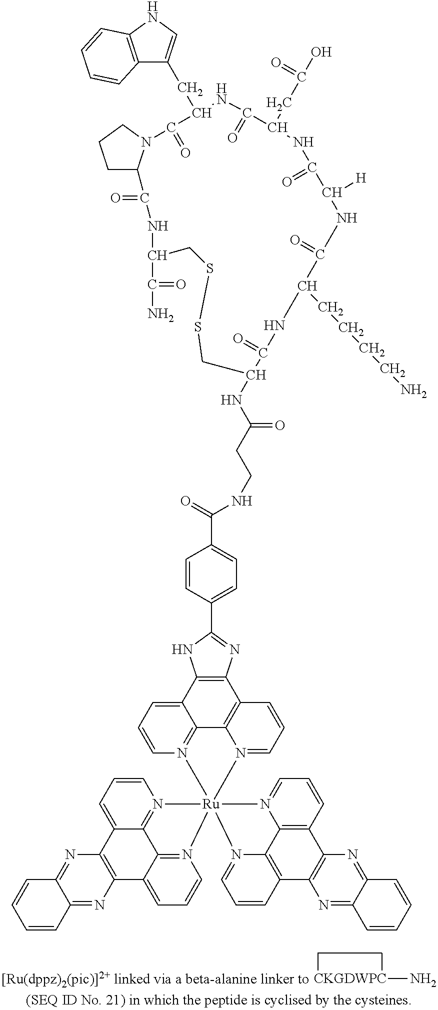

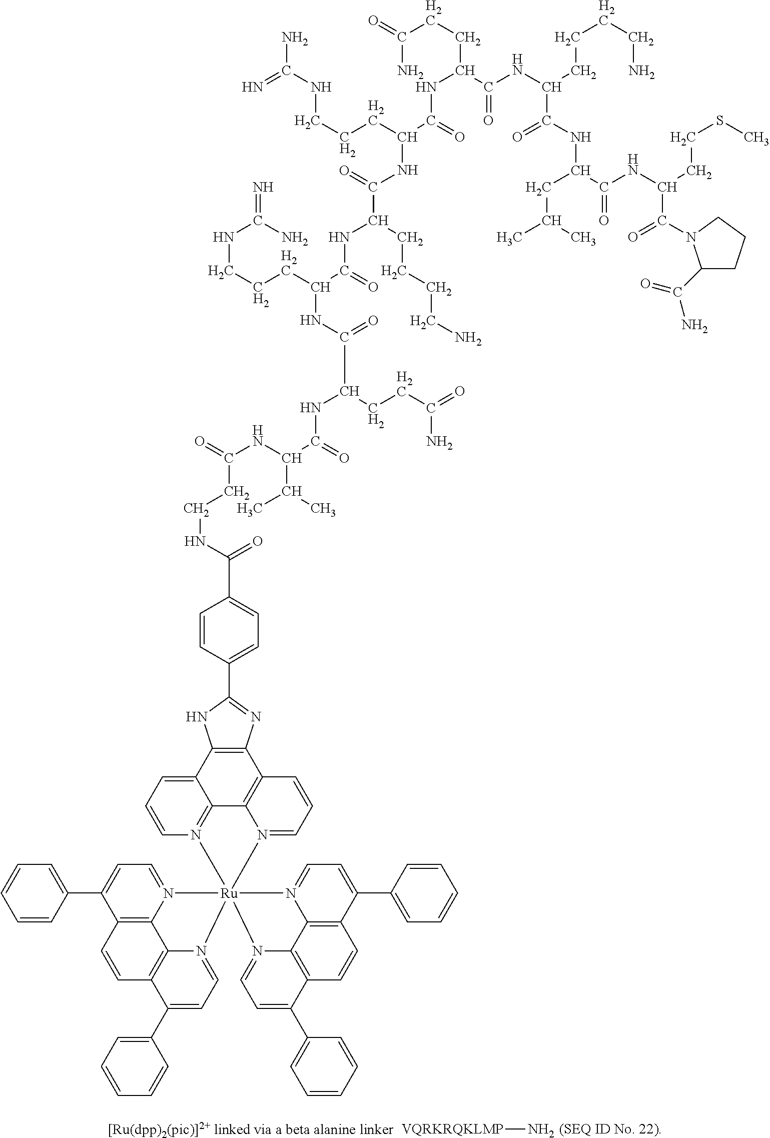

### Example 6

### Peptide Functionalisation of Ru^{II}(L¹)ₐ(L²)_{b}(PicH₂)²⁺

The Rₙ oligopeptides (n=5 or 8) were obtained by the Merryfield automated process, as a peptide immobilized on a polystyrene solid substrate. Then, the organic spacer was covalently linked to the peptide via conjugation of a pending amino group from the peptide with the carboxylic function of the 6-amino hexanoic acid spacer. Finally, the ruthenium complex Ru^{II}Ru(L¹)ₐ(L²)_{b}(PicH₂)²⁺ was attached to the resin immobilized molecule via another amide bond formation. In all cases, conventional reagents and catalysts were used to perform the syntheses. After the cleavage step in trifluoracetic acid, the final hybrid molecule was released from the polystyrene support, purified by HPLC and freeze-dried. Both Ru—R₅ and Ru—R₈ were characterized by MALDI-TOF mass spectrometry.

[Ru-LH]²⁺ possesses two ionisable sites at the imidazole, with pKas of 1.6 and 8.5, the deprotonation step results in an anionic charge at the imidazole. The complex exhibits a strong luminescence at 600 nm which is pH dependent, although resonance Raman and excited state pKa studies confirm that the excited state remains on the bipyridyl groups at all pHs.

### Example 7

### Resonance Raman Mapping

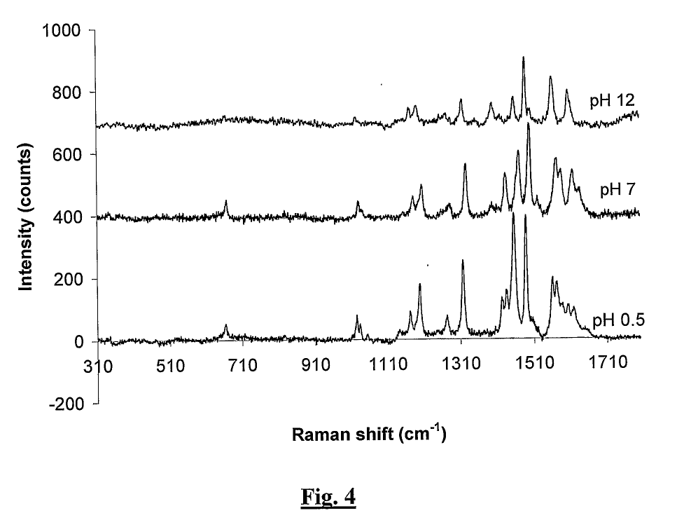

The complex Ru(L¹)ₐ(L²)_{b}(PicH₂)²⁺ exhibits a pH sensitive Raman profile, FIG. 4, and is an example of the type of complex which may be exploited for resonance Raman mapping/imaging of cells. By cells we mean nucleate formed cells and non-nucleate formed cells such as platelets. The cells may be living or dead.

Resonance Raman mapping/imaging has previously been used with cells which contain endogenous chromophores. We have demonstrated that resonance Raman imaging/mapping using exogenous dyes such as the dyes described herein can be used to report on the intracellular environment (e.g. pH or redox).

Resonance Raman imaging with the dye conjugates described herein works in two ways which may be separately or simultaneously exploited:
(a) A dyes absorbance changes with environment and therefore the resonant excitation line changes allowing for two colour resonance Raman mapping.
(b) The resonance Raman spectrum reports on structural changes in the dye which may be induced in a predictable by the environment. For example: pH, redox potential, etc.

The large Stokes shifts of these dyes means that when the dye is luminescent there is no luminescence interference and resonance Raman and fluorescence microscopy can be used together to provide complimentary imaging information.

The method is analogous to confocal fluorescence microscopy in the sense that exogenous dyes are introduced to target structural components and dynamic processes in chemically fixed as well as live cells and tissues. These dyes are chosen so that the dye absorbance is matched to the exciting laser wavelength. However, rather than fluorescence, this results in a large (up to 7 orders of magnitude) increase in the Raman intensity of the target dyes. The resonance Raman spectrum of the dye provides the vibrational modes of the chromophore, therefore structural insight into the dye and therefore information about the environment of the dye. For example, predictable changes to the dyes vibrational spectrum may occur with pH, ion binding or local redox potential. The distribution of dye and variation in its structure may then be imaged or mapped across cell or tissue using Raman spectroscopy.

Although resonance Raman imaging has been conducted using endogenous chromophores in cells, for example cytochrome C (Jan van Manen et al), to our knowledge, there have been no reports of resonance Raman imaging being used in conjunction with an exogenous probe that has been introduced into cells. In general, conventional cellular imaging dyes are unsuitable for this resonance Raman spectrum method as the small Stokes shift between their absorbance and luminescence will result in fluorescence interference. Dyes for resonance Raman imaging/mapping must either be non-luminescent or alternatively there must be a significant Stokes shift between their absorbance and luminescence. The metal based dyes described herein are luminescent and exhibit a suitable Stokes shift making them suitable for multi-modal imaging of cells and tissue.

Luminescence whilst not a prerequisite for the dyes used for resonance Raman imaging is advantageous as it allows for both techniques (resonance Raman imaging and fluorescence imaging) to be combined.

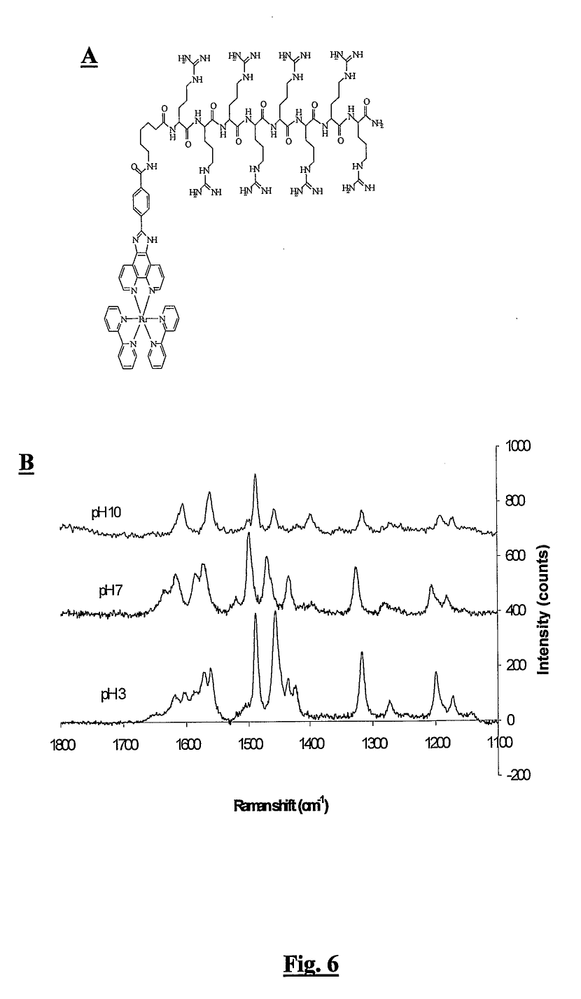

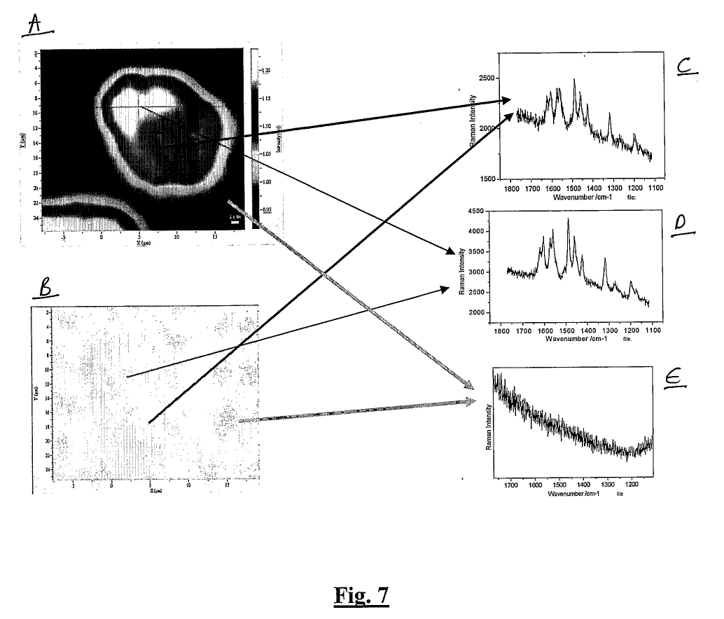

We used the octarginine labelled dyes as resonance Raman chromophores. These dyes exhibit pH dependent resonance Raman spectra exciting at 514 nm (FIG. 6). The resonance Raman mapping image of FIG. 7 was generated using vibrational mode at 1480 cm⁻¹ and the background around 1750 cm⁻¹ from the resonance Raman map of a myeloma cell. The distribution of the dye in the cytosol is seen, relatively little enters the nucleus or lies at the membrane and none is in the surrounding media. In addition, the pH lies between 6.4 and 7.2 throughout. pH can be mapped using a dye such as this whereby intensity (or area under) a pH insensitive and pH sensitive vibrational are ratioed. This can yield a pH titration for the dye, FIG. 16 B top, the ratio can then be used to map the regions of the cell of different pH, FIG. 16 B bottom, the pH of which can be obtained from the titration data.

Examples of redox probes include the oxygen nitrogen containing complex such as that shown below, (Keyes et al 1997 and 1998), which can be used, depending on the identity of metal and ligands to yield multiple spectral changes, in the redox range −0.2 to 1.3 V, with very distinct resonance Raman spectroscopies.

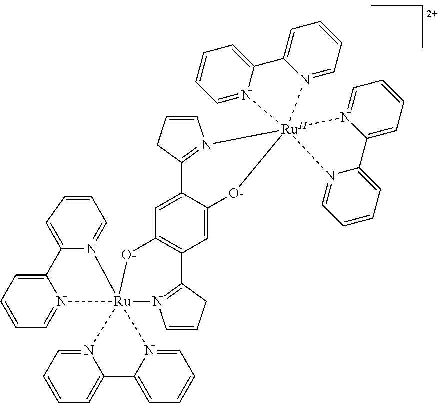

### Example 8

### Physical and Chemical Properties in Buffered Aqueous Solutions

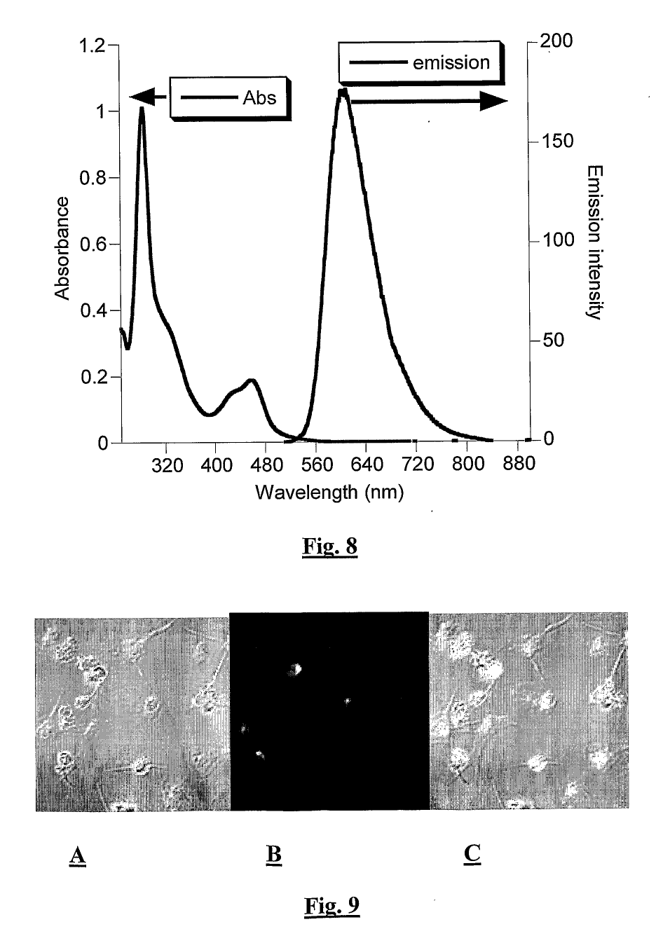

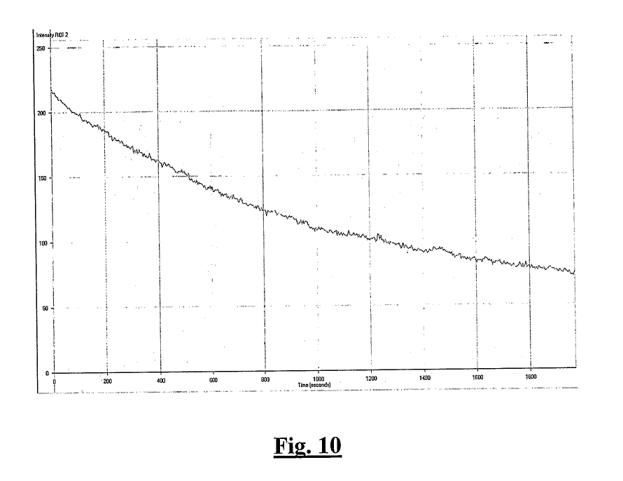

The photophysical properties of the complexes [Ru(bpy)₂(PicH-R8)]ⁿ⁺ have been investigated in pH 7 buffered aqueous solutions. The electronic absorption spectrum of the arginine derivatised complex in all cases is very similar to that of the parent complexes Ru(L¹)ₐ(L²)_{b}(PicH₂)²⁺. For example, for Ru(L¹)ₐ(L²)_{b}(PicH₂)²⁺ a strong absorption band at 460 nm is assigned to a metal to ligand charge transfer (FIG. 8). Excitation of the solution at this wavelength results in a strong luminescence at 607 nm. The quantum yield of the emission has been evaluated to 0.06, which is 30% higher than the case of the well-known complex [Ru(bpy)₃]²⁺. Lifetime measurements revealed that the luminescence of the complex is quite long-lived and oxygen sensitive, at 480 and 775 ns in aerated and deaerated solutions, respectively. The spectral features of complex Ru-Ahx-R₈ are very favourable for a luminescent tagging of platelets.

Fluorescence microscopy was performed on human blood platelets incubated in the presence of Ru-Ahx-R₅ and Ru-Ahx-R₈. The Ru-Ahx-R₅ compound showed no particular luminescence from within the cell, as is expected for this system as Ru-Ahx-R₅ is incapable of penetrating the platelets membrane. The Ru-Ahx-R₈ compound was transported through the cells membrane to accumulate intracellularly (FIG. 9). The process is largely irreversible, removing and washing cells, and resuspending in buffer results in less than 20% loss of emission intensity from the cell. The fluorescence microscopy pictures show intense, long-lived luminescence originating from the platelet; the wavelength of which (around 620 nm) can be assigned to the Ruthenium centres.

### Example 9

### Resonance Raman Imaging with [Ru(bpy)₂(PicH-R₈)]²⁺, [Ru(bpy)₂pic]²⁺, and [Ru(bpy)₃]²⁺

The Ru-dye [Ru(bpy)₂(PicH-R8)]ⁿ⁺ consists of a ruthenium metal centre, and three ligands: two bipyridine ligands and one picH ligand, where picH is 2-(4-carboxyphenyl)imidazo[4,5-f][1,10]phenanthroline. The octoarginine side chain is coupled to the carboxy end of the pic-ligand via an amide linkage. This peptide tail allows passive and efficient transport across the cellular membrane of living cells. Furthermore, the peptide-labelled dye exhibits very interesting photophysical properties which vary with pH and oxygen concentration in the environment.

UV/Vis Absorption

FIG. 13A shows the UV/vis absorption spectra of [Ru(bpy)₂(PicH-R8)]ⁿ⁺ in PBS at neutral pH (pH 7.4), and in acidic (pH 6.0 and 1.3) and basic (pH 8.4 and 10.2) solution. At around 458 nm, the dye exhibits a broad absorption band (εₙₑᵤₜᵣₐₗ˜16.9*10³ Lmol⁻¹ cm⁻¹) which can be assigned to the metal to ligand charge transfer transition (MLCT transition dπ-π*). At a shorter wavelength the ππ* transitions centred at the ligands are found: around 350 nm the ππ* transition at the pic ligand, and around 280 nm at the bipyridine ligands. As the pic ligand can be deprotonated at the NH of the imidazole ring, as well as protonated at the nitrogen at the same imidazole ring, the ππ* transitions of the pic ligand around 350 nm exhibits a strong pH dependence. The increasing absorption with increasing pH at 350 nm is plotted in FIG. 13B. Fitting this curve with a modified Henderson-Hasselbalch equation results in a pKₐ₁ of 1.72±0.07 and pKₐ₂ of 8.16±0.03. As expected the pH dependence of the other bands is rather minor.

### Luminescence

The Ru-arg8 complex exhibits a long lived luminescence in the red region (608 nm), which is shifted around 150 nm away from the absorption and well away from possible autofluorescence of biological material. The large Stokes Shift makes it possible to record resonance Raman spectra without interference from fluorescence when exciting in the MLCT band.

The luminescence wavelength and its intensity are pH dependent. While the emission maximum at pH 7 is at 608 nm, it shifts to higher wavelength with increasing and decreasing pH (λₘₐₓ (pH 1.3)=623 nm; λₘₐₓ (pH 11)=616 nm). When the pic ligand is in its neutral state, the luminescence is very bright (Φ=0.06), about 30% more intense than the one of [Ru(bpy)₃]²⁺. However, the quantum yield decreases with the pH moving away from the neutral pH, dropping to around half at basic pH and around 80% at acidic pH. When exciting into the isosbestic point of the absorption spectra and fitting the plot of the luminescence intensity as a function of pH, the excited state pKa was determined to be pKa₂*=8.1±0.2 which is very similar to the ground state pKa obtained from the UV/vis absorption data, confirming in this case that the excited state rests on the bpy ligands.

### Luminescence Lifetime

As is typical for ruthenium complexes the lifetime of [Ru(bpy)₂(PicHR₈)]ⁿ⁺ is several hundred nanoseconds. As shown for the luminescence intensity, the lifetime can be altered by changing the pH and is quenched by the presence of oxygen.

pH dependence: The lifetime of [Ru(bpy)₂(PicHR₈)]ⁿ⁺ exhibits sensitivity to pH in PBS buffer, FIG. 14A, when the PicH moiety is in its neutral state, at pH 6, a luminescence lifetime τ of τ=860 ns is observed. Upon deprotonation of the imidazole of the pic moiety the lifetime drops down to τ=521 ns at pH 11 in degassed PBS (FIG. 14B). The change of lifetime with pH was fitted with a sigmoidal

$${τ}_{0} = 860 ns - \frac{( 860 - 521 ) ns}{1 + exp ( \frac{pH - 8.2}{0.62} )} .$$

This curve can be used to obtain the unquenched lifetime for a known pH.

O₂ dependence: The luminescence of [Ru(bpy)₂(PicH-R8)]²⁺ shows strong dependence on concentration of molecular oxygen. In air saturated PBS solutions the lifetime drops down to 540 ns when the pic ligand is neutral, and even down to 350 ns, when the pic moiety is deprotonated. Stern-Volmer plots are linear up to air saturated solutions and then start levelling of. The Stern-Volmer quenching constants range actually vary very little with pH 2100 M⁻¹ for pH 10 to around 2900 M⁻¹ when the pic moiety is in its neutral state. The corresponding bimolecular rate constants vary between around 3.6×10⁻⁹ M⁻¹ s⁻¹ when the pic moiety is deprotonated to 3.0×10⁻⁹ M⁻¹ s⁻¹ when the pic moiety is in its neutral state, which is essentially the same within experimental error. This means that within the limited range of the cellular environment the O₂ concentration within a cell can be assessed independently of pH using this dye.

Resonance Raman spectroscopy

Resonance Raman spectra are recorded by exciting in the MLCT transition band at 458 nm. Due to the resonance effect dye concentrations as low as 1.2 μM can be used to obtain Raman spectra with good signal-to-noise ratio.

The resonance Raman spectrum of [Ru(bpy)₂(piCH₂)]²⁺ exhibits spectral features of both ligands, the bipyridine and the pic, as can be easily seen in FIG. 15A by comparing the resonance Raman spectrum of [Ru(bpy)₂(piCH₂)]² (middle) with the ones of the two homoleptic complexes [Ru(bpy)₃]²⁺ (bottom) and [Ru(piCH₂)₃]²⁺ (top). Vibrational bands assigned to the bipyridine ligand are observed at 1603, 1559, 1486, 1314, 1269, 1172, 1024, and 664 cm⁻¹. The Raman bands which originate mainly from vibrations of the pic moiety appear to be slightly weaker, but still significant. The most isolated ones can be found at 1625 and 1509 cm⁻¹. Other prominent bands such as at 1457 and 1422 cm-1 do not have any correspondent in the homoleptic complexes. Their occurrence can be explained with a change in symmetry in the mixed complex and the bands can be assigned to pic bands (maybe due to post-resonance of the pic ligand's ππ*-transition around 350 nm) with bpy contribution.

As the pH changes, the pic moiety can be protonated or deprotonated, while the bpy moieties should stay mainly unaffected by changes in pH. Therefore, [Ru(bpy)₂(PicHR₈)]ⁿ⁺ is ideally suited to probe the pH in the environment, because it possesses intrinsically a Raman standard (bands from the bpy moiety). Resonance Raman spectra of [Ru(bpy)₂(piCH₂)]² at different pH are shown in FIG. 15B. While the Raman bands around 1485 and 1314 cm⁻¹ (bpy bands) stay unchanged over the whole pH range, a shoulder at 1625 and at 1575 cm⁻¹ (pic bands) evolve as the pH is decreased from around pH 11. Therefore, the ratio of the changing peaks with the unchanging peaks will give a measure of the protonation state of the pic moiety in the complex and therefore, can be used to obtain the pH in the solution. Such a plot is shown in FIG. 16B for the ratio of the Raman peaks at 1575 and 1317 cm⁻¹. To simplify and allow automation of the data analysis, the area under the Raman band of interest was used, instead the exact deconvoluted peak intensity. The change of the ratio with pH was fitted with Henderson-Hasselbalch equation and gave a pKa of 8.5±0.2.

At pH 0.5, the spectrum is similar, with contributions from both picH and bpy evident, but in addition, new features appear at 1588, 1436 and 1055 cm⁻¹. This suggests that at low pH, the extent of delocalisation of the picH(π*) state across the ligand has increased.

An absorbance band centred around 450 nm contains contributions from both Ru(dπ) to bpy(π*) and Ru(dπ) to picH(π*) MLCT underlying transitions. At this pH, ligand-based absorption bands have moved out.

### Incorporation and Localization of the [Ru(bpy)₂(PicH-R8)]²⁺ Dye Inside Mammalian Cells

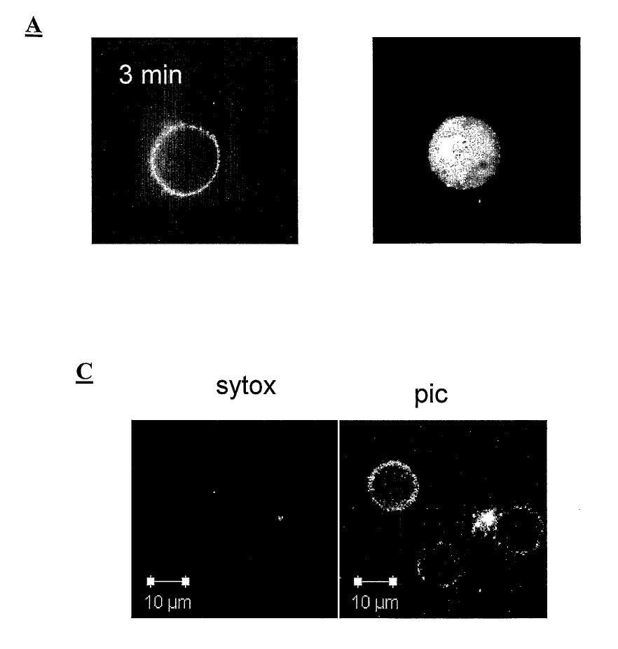

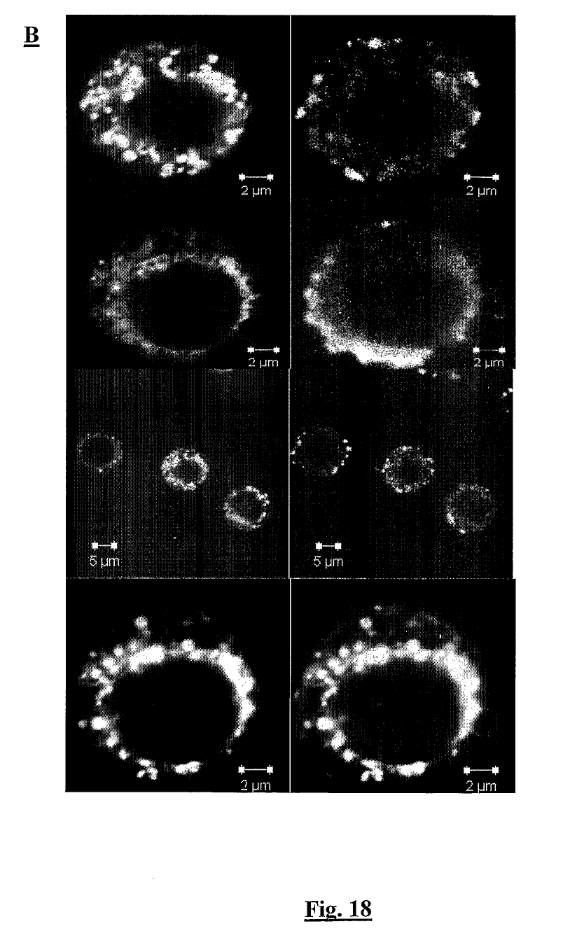

The octoarginine was shown previously to enter passively into mammalian cells (Example 7). Penetration is fast and complete after about 15 to 20 minutes. In the first few minutes the dye is penetrating the cellular membrane and a bright fluorescence lights up the membrane around the cell (FIG. 18A). From here, the dye distributes into and throughout the cell with slight intensity variations across the cellular plasma membrane. After even longer incubation time (>40 min) the dye was found to localise in the nucleus as well.

The commercial dye DiOC6(3) is known to stain mitochondria and at higher concentration membrane structures in the cell, such as endoplasmatic reticulum. Counterstain experiments were performed to localize the Ru-complex. As can be seen in FIG. 18B, the [Ru(bpy)₂(PicH-R₈)]ⁿ⁺ complex has no special preferences for mitochondria. If however a localising peptide such as the peptide of SEQ ID No. 1 was conjugated to the dye complex, the dye would be specifically targeted to mitochondria.

Counterstaining with Sytox green fulfilled two purposes, first to assess that the Ru-stained cells were still viable, as Sytox green only penetrates cells with compromised plasma membranes and yet would not cross the membranes of live cells (FIG. 18C) and second, after killing/permeabilising the Ru-stained cells, it was used to identify the nucleus. The experiments confirmed that the [Ru(bpy)₂(PicHR₈)]ⁿ⁺ did not kill the cells over the time period investigated, over 2 hours.

### Probing pH Inside Living Cells

FIG. 16A shows the greyscale Raman map of a myeloma cell incubated with the [Ru(bpy)₂PicH-R₈]ⁿ⁺ dye for 15 min, using the most intense Raman band of the dye centered at 1487 cm⁻¹. This band is not sensitive to changes in pH and therefore can be used to monitor the distribution of the dye inside the cell. As already seen in the localization experiments, the dye is distributed throughout the cytoplasm, with less dye in the nucleus (darker spot on the bottom part of the cell). Modelling the spectra, areas with very similar spectra were grouped together as can be seen in the greyscale map. (FIG. 16A (left)). The averaged resonance Raman spectra from the different regions inside the cell are shown in FIG. 16A (far left). No dye is found outside the myeloma cells as the cells were washed twice with PBS after incubation with the dye. The spectra from inside the cells were used to determine the ratio of the pH dependent peaks and the pH-independent peaks. This ratio was used to estimate the pH in the different compartments inside the cell using the calibration plot as depicted in FIG. 16C. For the outer cellular layer (membrane) a pH of 7.7 was estimated, for the cytoplasm pH 7.85 and for the nucleus pH 7.0. When using an average of the resonance Raman spectra from the whole cell (all areas) a pH of 7.5 was determined. The estimated pH from the experiment is in good agreement with what is currently known about the pH in living cells. It was also demonstrated that by changing the pH of the external medium, for example using PBS or Dulbecco's medium, resonance Raman imaging could detect these changes within the cytoplasm.

### Probing O₂ Concentration Inside Living Cells

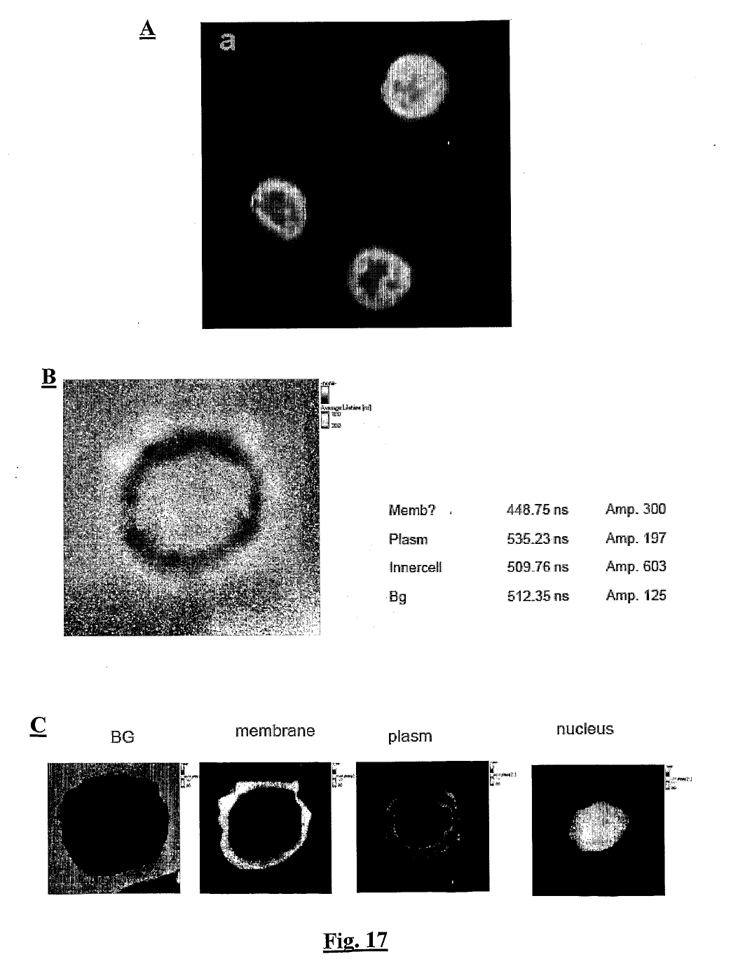

According to the calibration plot of FIG. 14B (left) a pH of 7.5 corresponds to a luminescence lifetime of 451 ns in air saturated PBS, and to 779 ns in degassed aqueous solution. FIG. 17A shows the fluorescence intensity map of a myeloma cells incubated for 12 minutes with the Ru-arg8 dye, and FIG. 17B shows a greyscale lifetime image of one of these cells. By selecting different regions, similar to the ones obtained from the Raman modelling, FIG. 17 C, we obtained a lifetime of 513 ns for the background, 452 ns for the cell membrane region, 540 ns for the cytoplasm, and 709 ns for the nucleus. Which yields O₂ concentrations of saturation O2 from the membrane, (approx 8.5 mg/L) approx 4.5 mg/L for the cytoplasm and approx 1 mg/L from the nucleus.

We have demonstrated that dye-peptide conjugates can be used to probe the pH and O₂ concentration inside living cells which are otherwise difficult to measure. These two parameters are very significant for the cellular metabolism and could be used as informative marker for the activity of cellular processes or tumour development.

As a probe, a family of peptide labelled ruthenium-complexes are used. These complexes contain three ligands which can be functionalized to fulfil different functions such as transport into the cell, target a certain feature inside the cell or sub-cellular structure and report on cellular environmental conditions. These dyes can be used to independently report on two different parameters through emission and resonance Raman microscopies. We have demonstrated that using a single dye that has been transported inside a living cell, resonance Raman mapping/imaging can be used to report on the pH of the intracellular environment and fluorescence lifetime imaging can be used to obtain information on the intracellular O₂ concentration. We have also demonstrated that a water sensitive dye can be used to image membranes within a cell using fluorescence microscopy and resonance Raman mapping/imaging can be used to determine the distribution of the dye throughout the cell. The dual functionality of the dyes (luminescence and fluorescence) is advantageous as it means that two parameters can be imaged/mapped simultaneously within a single cell thereby providing detailed information, and in some circumstances real time data, of an intracellular environment.

Resonance Raman and fluorescent lifetime measurements can be made without altering/compromising the metabolism or viability of a cell. The peptide chain (such as R₈) allows for passive transport across the membrane, so that the dye can be rapidly transported inside living cells without affecting the viability of the cells as was demonstrated by the trypan blue and Sytox green staining experiments.

To our knowledge, this is the first time resonance Raman imaging using an exogenous dye has been used to record cellular properties and to provide information on cellular microenvironments and to locate an environmentally sensitive dye in a cell. The use of resonance Raman spectroscopy makes it possible to detect very low concentrations of dye, which are suitable for fluorescence imaging within the cell, indeed the same cells were used for both techniques. Furthermore, the technique can be combined with fluorescence lifetime measurement to obtain additional quantitative information. The emission of the Ru-dye lies in the red wavelength region, which is spectrally far away from possible autofluorescence of biological material. The high quantum yield permits easy detection, even at low dye concentration. For these reasons, both of these techniques are ideally suited to biological applications. Resonance Raman mapping and fluorescence lifetime imaging experiments may be performed on the same cell, ideally using an instrument which can record both luminescence and fluorescence information simultaneously.

Fitting the luminescence lifetime of the Ru-dye at different pHs, when inside the myeloma cells to a monoexponential decay is a simplification of the real model. However, it was shown for the pure dye at different pH, that a fit with two components with fixed lifetimes of the pure species did not improve the quality of the fit significantly. Furthermore, the average and mean lifetime obtained from the biexponential fit follows quite closely the lifetime obtained from the monoexponential fit. The change of the unquenched lifetime in degassed PBS with pH was fitted with a sigmoidal

$${τ}_{0} = 860 ns - \frac{339 ns}{1 + exp ( \frac{pH - 8.2}{0.62} )} . \qquad ( Fig . 16 )$$

This curve can used to obtain the unquenched lifetime for a known pH.

The long-lived luminescence of the dye may be well suited to explore some of the longer lived, microsecond biodynamical processes in real time such as membrane diffusion, protein rotation or folding.

### Example 10

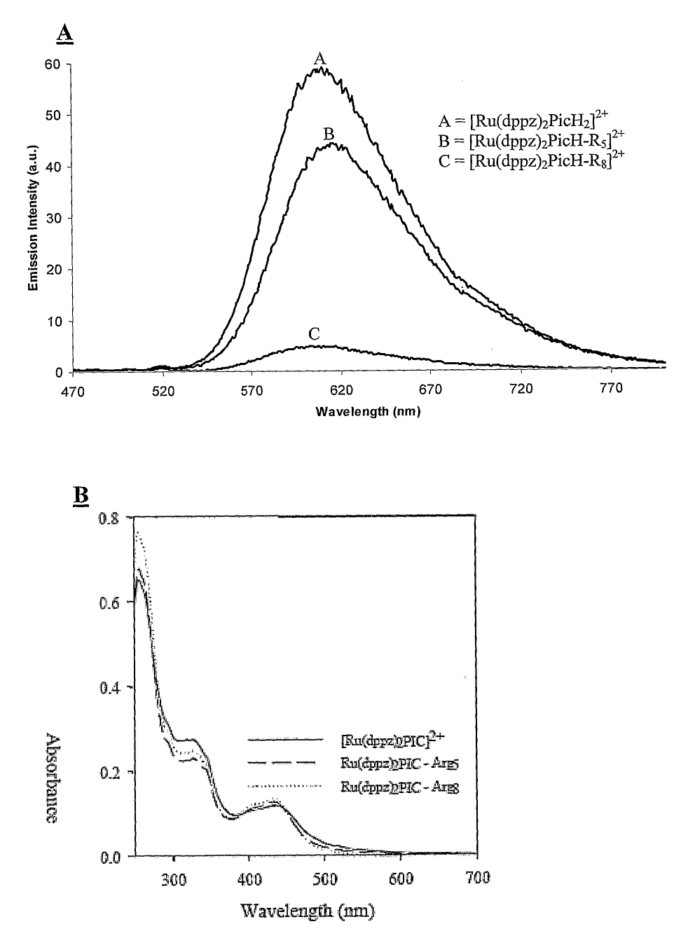

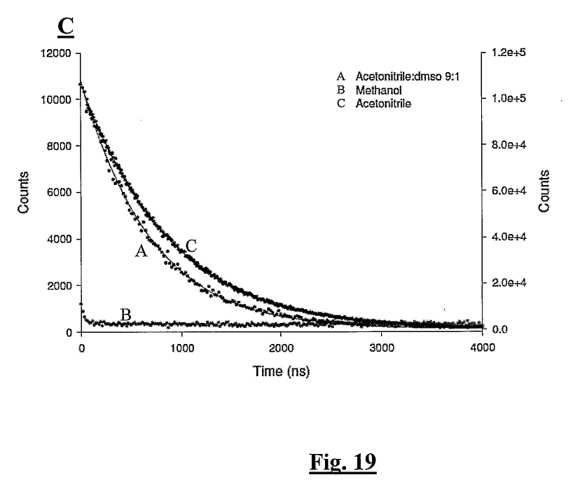

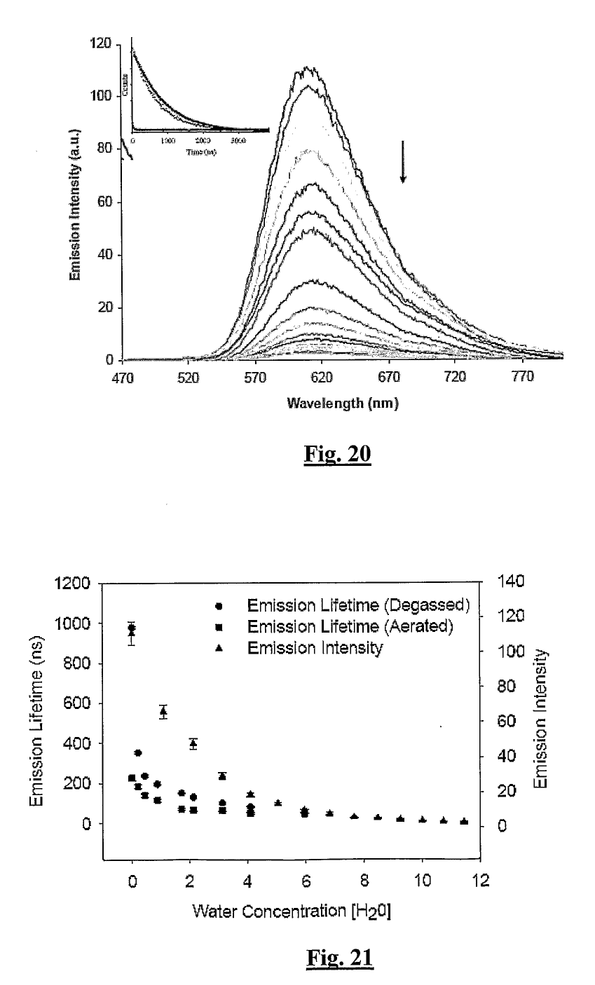

Use of [Ru(dppz)₂PicH-R₈]ⁿ⁺ and [Ru(dppz)₂PicH-R₅]ⁿ⁺ as Cellular Probes

[Ru(dppz)₂PicH-R₈]ⁿ⁺, FIG. 11 C, was prepared as a membrane probe. Its photophysical data along with that of the parent complex without peptide are shown in FIG. 11 and FIG. 19. The complex is readily quenched in protic solvent such as methanol, FIG. 19C and as shown in the emission data FIG. 20 and lifetime data (FIG. 21) is strongly quenched by water, FIGS. 11 A and B.

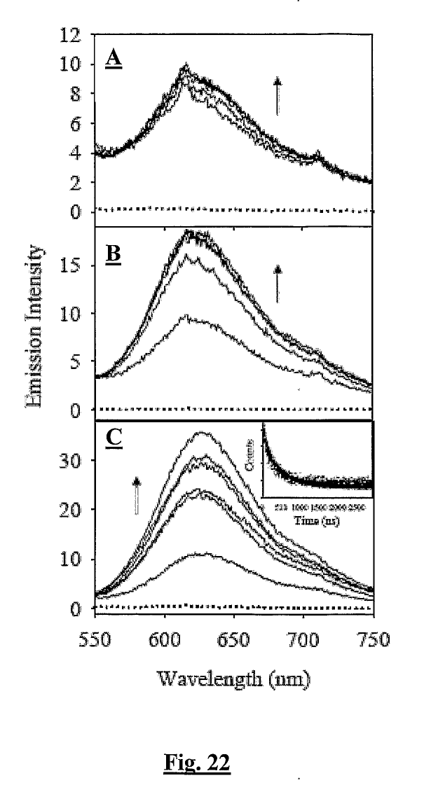

In order to investigate the membrane-crossing ability of the labelled peptides, and whether they are luminescent in membranes, DPPG (Dipamitoylphosphatidylglycerol) liposomes were employed to mimic the phospholipid bilayer of cells. The luminescence intensity of the dye and the peptide conjugate with 5 and 8 arginines were compared in water (where it does not luminesce) in the presence of the liposomes was monitored over time, FIG. 22 A to C, with the inset in C showing the luminescent lifetime of [Ru(dppz)₂PicH-R₈]ⁿ⁺ in liposome. In all instances the dyes became luminescent in the presence of the liposomes, suggesting this is a useful membrane probe. In each instance, the material does not luminesene in the absence of liposome.

After 24 hours [Ru(dppz)₂PicH-R₅]ⁿ⁺ and [Ru(dppz)₂PicH-R₈]ⁿ⁺ showed comparable emission intensity, whereas [Ru(dppz)₂PICH₂]²⁺ showed double the intensity (dye and liposome concentrations and quantum yields are expected to be the same). This is most likely due to the smaller size of [Ru(dppz)₂PicH₂]²⁺ in comparison with [Ru(dppz)₂PicH-R₅]ⁿ⁺ and [Ru(dppz)₂PicH-R₈]ⁿ⁺. This would allow a higher concentration of intercalated fluorophore over the surface area of the liposome.

Table 1 describes the luminescent lifetimes for the dyes in liposome and non-aqueous solvent. For the liposomes biexponential luminescent decays are observed

TABLE 1
Emission lifetime comparisons between
liposomes and free solution

| | τ (μs) in DPPG | | |
|---|---|---|---|
| | liposome in PBS buffer | | τ (μs) in |
| | Biexponential decay | | acetonitrile:DMSO |
| Material | τ₁ | τ₂ | 9:1 v/v |
| [Ru(dppz)₂PICH₂]²⁺ | 0.36 | 0.1 | 0.68 |
| [Ru(dppz)₂PicH—R₅]ⁿ⁺ | 0.50 | 0.08 | 0.75 |
| [Ru(dppz)₂PicH—R₈]ⁿ⁺ | 0.50 | 0.08 | 0.78 |

Table 2 below examines the O₂ sensitivity of the dye labelled peptides in liposomes. In the membrane environment the dyes show no O₂ sensitivity within experimental error.

TABLE 2
Emission lifetimes recorded in DPPG liposome
(1 mg/mL) at pH 7.4 in PBS buffer

| | τ (μs) in liposome | | τ (μs) in liposome | |
|---|---|---|---|---|
| | Biexponential decay | | Biexponential decay | |
| Material | τ₁ | τ₂ | τ₁ | τ₂ |
| [Ru(dppz)₂PICH₂]²⁺ | | | | |
| [Ru(dppz)₂PicH—R₅]ⁿ⁺ | 0.49 | 0.07 | 0.53 | 0.08 |
| [Ru(dppz)₂PicH—R₈]ⁿ⁺ | 0.49 | 0.08 | 0.49 | 0.07 |

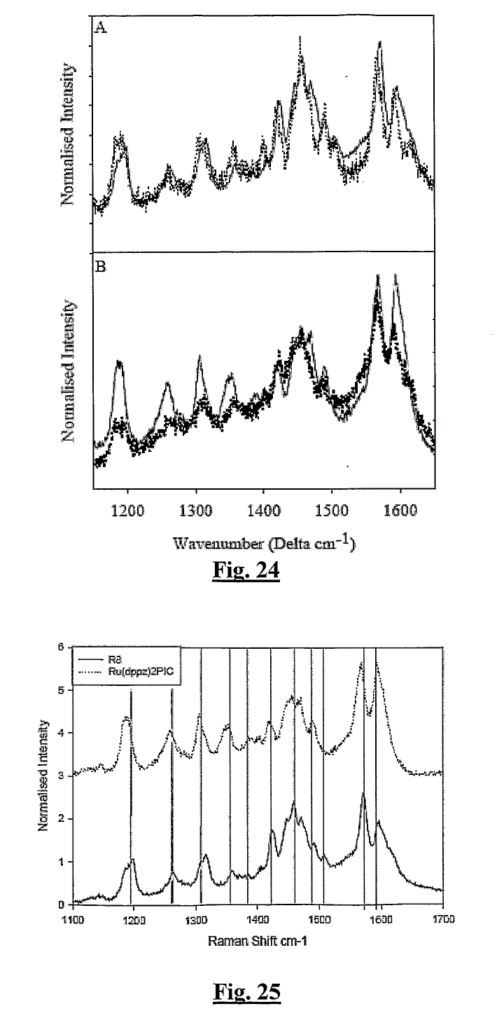

The utility of [Ru(dppz)₂PicH-R₈]ⁿ⁺ as a cellular probe was investigated using mouse SP2/0 myeloma cells. Concentrated solutions of [Ru(dppz)₂PicH-R₈]ⁿ⁺ and [Ru(dppz)₂PicH₂]²⁺ were prepared at pH 7.4 phosphate buffered saline. The complexes were then mixed with myleoma cells, after which the cells were washed with PBS. As [Ru(dppz)₂PicH-R₈]ⁿ⁺ and [Ru(dppz)₂PicH₂]²⁺ only emit light when in the membrane, fluorescence microscopy for both complexes with the cell looked quite similar both showed emission from the membrane, FIG. 26. However, it was not possible from fluorescence microscopy to determine if both had in fact transferred across the membrane. This was where resonance Raman mapping became very useful. FIG. 25 shows the resonance Raman spectroscopy of micromolar concentration of [Ru(dppz)₂PicH-R₈]ⁿ⁺ in aqueous buffer excited at 458 nm, a strong resonance Raman signal was observed for the dye. Resonance Raman mapping of the individual myleoma cells is shown in FIG. 23 following incubation with [Ru(dppz)₂PicH₂]²⁺ (top) and [Ru(dppz)₂PicH-R₈]ⁿ⁺ (bottom) white light images are shown to the left. Representitive resonance Raman spectra used to yield the image are shown in FIGS. 24 and B. Resonance Raman imaging confirmed irrefutably that whereas Ru(dppz)2PICH-Arg8 entered the cell and distributed into the cytoplasm, [Ru(dppz)₂piCH₂]²⁺ only interacted with the membrane but did not cross it. This is likely to be due to intercalation of the dppz ligands into the cell membranes. The highest emissions (and resonance Raman) signals were observed between cells where two adjacent membranes may allow for better protection of both dppz ligands.

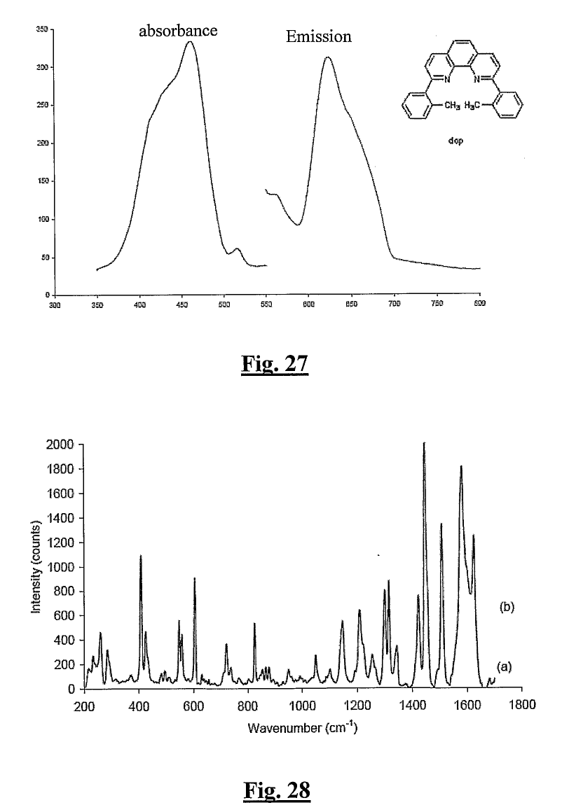

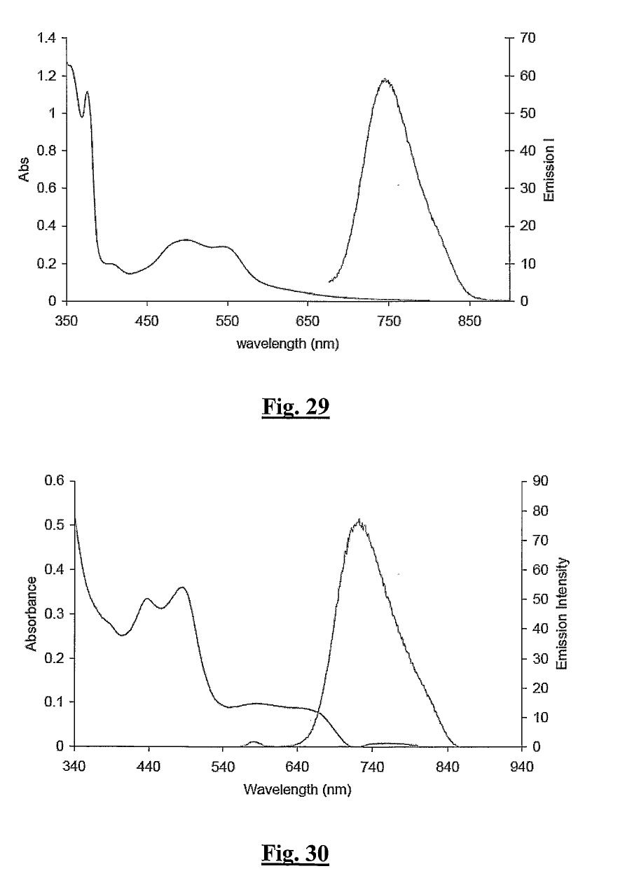

FIGS. 27 to 30 show spectroscopic properties of other metal complexes which have appropriate, luminescence, stokes shifts and resonance Raman spectroscopy to be valuable in the applications described here.

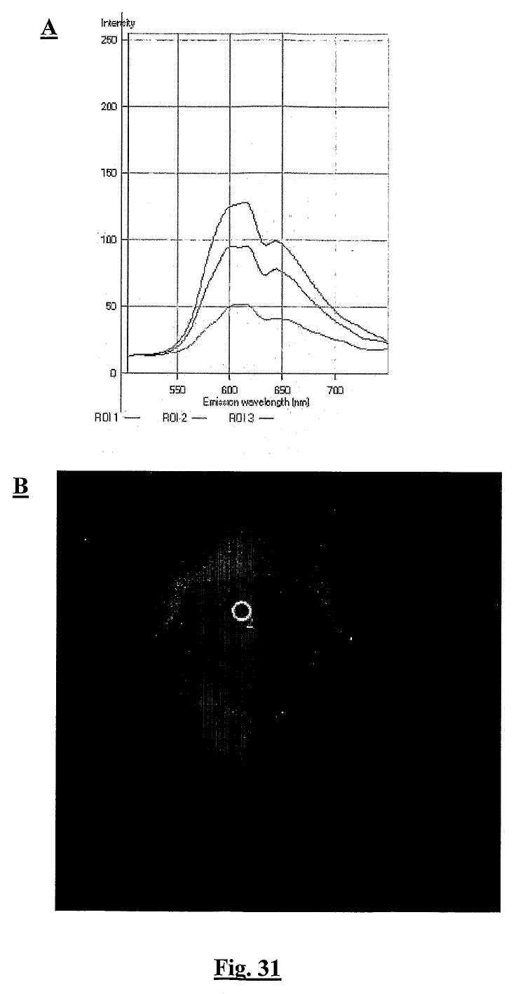

FIG. 31 shows an exemplary of the application of an alternative peptide label on piCH₂[Ru(bpy)₂(pic-KVG)]ⁿ⁺ wherein KVG is Lys-Val-Arg. Here it has been used to image emission spectrum (left) and a confocal image at a excitation wavelength of 458 nm (right) of bovine aortic epithelial (BAE) cells. The dyes were incubated at 22° C. with BAE cells in PBS buffer. The luminescence image suggests that the dyes concentrates in the membrane, which is consistent with its binding to integrin protein within the membrane.

The invention is not limited to the embodiment hereinbefore described, with reference to the accompanying drawings, which may be varied in construction and detail.

### REFERENCES

S. Bodige, F. M. MacDonnell, Tetrahed. Lett., 1997, 38, 8159-8160
F. Charbonnier, S. Penades, Eur. J. Org. Chem., 2004, 3650
O. C. Colgan, G. Ferguson, N. T. Collins, R. P. Murphy, G. Meade, P A. Cahill, P. M. Cummins Am J Physiol Heart Circ Physiol 292:3190-3197, 2007.
R. J. Edwards, N. Moran, M. Devocelle, A. Kiernan, G. Meade, W. Signac, M. Foy, S. D. Park, E. Dunne, D. Kenny and D. C. Shields, Nat Chem Biol Vol: 3 Issue: 2 Pages: 108-12
Fischer R. et al, Bioconjugate Chem 2003, 14, 653-660
E. A. Goun, R. Shinde, K. W. Dehnert, A. Adams-Bond, P. A. Wender, C. H. Contag, B. L. Franc, Bioconjugate Chemistry 2006, 17 (3), 787-796.
H. Jan Van Manen et al, J. Am. Chem. Soc. 2003, 125(40), 12112-12113
T. E. Keyes, P. M. Jayaweera, J. J. McGarvey and J. G. Vos J. Chem. Soc. Dalton Trans., 1997, 9, 1627
T. E. Keyes, R. J. Forster, P. M. Jayaweera, J. G. Vos, J. J. McGarvey Inorg Chemistry, 22, 5925 1998
T. E. Keyes, D. Leane, R. J. Forster, C. G. Coates, J. J. McGarvey, J. G. Vos, Inorg. Chem., 41, (22) 5721, 2002
Kiernan, G. Meade, W. Signac, M. Foy, S D E Park, E. Dunne, D. Kenny, D C Shields, Nature, Chem. Biol. 3, 108-112 (2007).
M. Kunishima, C. Kawachi, J. Morita, K. Terao, F. Iwasaki, S. Tani, Tetrahedron, 1999, 55, 13159.
E. A. Medlycott, G. S. Hanan, Coord. Chem. Rev. 2006, 250, 1763-1782. V. Balzani, P.
X. Michalet, F. F. Pinaud, L. A. Bentolila, J. M. Tsay, S. Doose, J. J. Li, G. Sundaresan, A. M. Wu, S. S. Gambhir, S. Weiss, Science, 2005, 307.538-544.
Moran et al. N. Moran, A. Kiernan, E. Dunne, R. J. Edwards, D. C. Shields and D. Kenny, Analytical Biochemistry, 357, 1, 77-84
M. C. Morris, J. Depollier, J. Mery, F. Heitz, G. Divita, Nature Biotechnology 2001, 19, 1173-1176
Raymo et al, Phys. Chem. Chem. Phys, 2007, 9 2036-2043
G. Ruan, A. Agrawal, A. M. Smith, X. Gao S, Nie, in Reviews in Fluorescence, 2006, 8, 181-193
A. T. R. Williams, S. A. Winfield and J. N. Millar: Analyst 108, 1067, (1983).
Wang et al, JACS 2007 129(14), 3848-3856
Org Biomol Chem. 2008 Jul. 7; 6(13):2242-55
Nature Medicine, 10(3):310-315 (2004)
Chemistry & Biology, (8):943-948 (2002)
Biochem. J. (2006) 399, 1-7
Expert Opinion on Pharmacotherapy, 653-663, 7(6), 2006
Expert Opinion on Investigational Drugs, 933-946, 15(8), 2006
Anti-Cancer Agents in Medicinal Chemistry, 2007, 7, 552-558
Arch. Immunol. Ther. Exp. 2005, 53: 47-60
Nature Chemical Biology 2007 3(2):108-112
Biochemistry 2006 45: 12295-12302

1-38. (canceled)

39. A use of a metal complex having the formula:
[M(L¹)ₐ(L²)_{b}(L³)_{c}]-X_{d}-Pepₑ
wherein:
M is a metal selected from osmium, ruthenium, rhodium, rhenium or copper;
L¹, L², L³ are bidentate or tridentate heterocyclic ligands containing O and/or N and may be the same or different;
a, b, c are integers between 0 to 3 and may be the same or different and wherein the sum of a+b+c is 2 or 3;
X is a functional group for directly or indirectly covalently binding to Pep wherein the functional group for directly covalently binding to Pep is selected from: amine, carboxylic acid, thiol or azide reactive functionalities;
Pep is a peptide;
d and e are integers between 1 and 3 and are the same and wherein the integers for d and e are equal to or less than the sum of a+b+c; and
wherein there is optionally a linker between X and Pep
for imaging of a cell or cell derived biological sample.

40. The use as claimed in claim 39 for resonance Raman imaging and/or mapping of a cell or cell derived biological sample.

41. The use as claimed in claim 39 for fluorescent imaging of a cell or cell derived biological sample.

42. The use as claimed in claim 39 for resonance Raman imaging and/or mapping and fluorescent imaging of a cell or cell derived biological sample.

43. The use as claimed in claim 41 wherein the fluorescent imaging is fluorescent lifetime imaging.

44. The use as claimed in claim 39 wherein one or more of L¹, L², L³ is selected from the group comprising: 2,2-bipyridyl (bpy), 2,2-biquinoline (biq), 4,7-diphenyl-1,10-phenathroline (dpp), 2,3-bis(2-pyridyl)pyrazine (dppz) and 2-(4-carboxyphenyl)imidazo[4,5-f][1,10]phenanthroline (piCH₂).

45. The use as claimed in claim 39 wherein the group to provide amine functionality is selected from one or more of carboxylate, active ester, acid halide or isothiocyanate functionalities.

46. The use as claimed in claim 45 wherein the active ester is a succinimidyl ester and/or a hydroxybenzotriazole ester.

47. The use as claimed in claim 39 wherein the group to provide carboxylic acid functionality is selected from one or both of amine or isothiocyanate functionalities.

48. The use as claimed in claim 39 wherein the group to provide thiol functionality is selected from one or more of iodoacetamide, maleimide, alkyl halide or isothiocyanate functionalities.

49. The use as claimed in claims 39 wherein the group to provide azide reactive functionality is an alkyne functionality.

50. The use as claimed in claim 39 wherein the peptide comprises up to 50 amino acids in length.

51. The use as claimed in claim 39 wherein the peptide comprises up to 30 amino acids in length.

52. The use as claimed in claim 39 wherein the peptide comprises up to 20 amino acids in length.

53. The use as claimed in claim 39 wherein the peptide comprises up to 10 amino acids in length.

54. The use as claimed in claim 39 wherein the peptide comprises a transmembrane delivery sequence.

55. The use as claimed in claim 39 to wherein the peptide comprises any one of the amino acid sequences of SEQ ID No. 1 to SEQ ID No. 22.

56. The use as claimed in claim 39 wherein there is a linker between X and Pep.

57. The use as claimed in claim 56 wherein the linker is an aliphatic compound.

58. The use as claimed in claim 57 wherein the linker comprises an aliphatic compound having at least 2 carbon atoms.

59. The use as claimed in claim 57 wherein the linker comprises an aliphatic compound having from 2 to 10 carbon atoms.

60. The use as claimed in claim 56 wherein the linker is saturated.

61. The use as claimed in claim 56 wherein the linker comprises a functional carboxyl group.

62. The use as claimed in claim 56 wherein the linker is a straight chain molecule.

63. The use as claimed in claim 56 wherein the linker is a hexyl linker.

64. The use as claimed in claim 56 wherein the linker is a beta alanine.

65. The use as claimed in claim 39 wherein the cell or cell derived biological sample comprises live cells.

66. The use as claimed in claim 39 wherein the cell or cell derived biological sample comprises a tissue sample.

67. The use as claimed in claim 39 wherein the metal complex has a Stokes shift of at least 50 nm.

68. The use as claimed in claim 39 wherein the metal complex has a Stokes shift of at least 100 nm.

69. The use as claimed in claim 39 wherein the metal complex has a Stokes shift of at least 150 nm.

70. The use as claimed in claim 39 wherein the metal complex is luminescent.

71. The use as claimed in claim 39 wherein the metal complex has an excitation wavelength between 380 nm to 1300 nm.

72. The use as claimed in claim 39 for imaging environmental parameters of a cell or cell derived biological sample.

73. The use as claimed in claim 72 wherein the environmental parameters are selected from one or more of oxygen concentration, pH, and redox state.

74. The use as claimed in claim 73 wherein the oxygen concentration of a cell or cell derived biological sample is imaged using fluorescence lifetime imaging.

75. The use as claimed in claim 73 wherein the pH of a cell or cell derived biological sample is imaged using resonance Raman imaging and/or mapping.

76. The use as claimed in claim 73 wherein the redox state of a cell or cell derived biological sample is imaged using fluorescence lifetime imaging and/or resonance Raman imaging and/or mapping.
